(19) United States
(12) Patent Application Publication
(10) Pub. No.: US 2005/0282781 A1
(43) Pub. Date: Dec. 22, 2005

(54) Compositions of stable bioactive metabolites of docosahexaenoic (DHA) and eicosapentaenoic (EPA) acids

(76) Inventor: Shibnath Ghosal, West Bengal State (IN)

Correspondence Address: Diane Dunn McKay, Esq.;Mathews, Collins, Shepered & McKay, P.A., Suite 306, 100 Thanet Circle, Princeton, NJ, 08540, US

(21) Appl. No.: 10/872,276

(22) Filed: Jun. 18, 2004

(51) Int. Cl. A61K 31/366; A61K 31/665

(52) U.S. Cl. 514/80; 514/454

(57) ABSTRACT

An invention that adduces cogent evidence to establish that oxygenated dibenzo-α-pyrones (DBPs and their conjugates), the major bioactives of shilajit (Ayurvedic vitalizer), have their origin, at least partly, in EPA and DHA. Earlier research has shown that, in mammals, C-20 PUFAs are metabolized by oxygenases and other enzymes to produce short-lived prostaglandins, leukotrienes and thromboxanes that bind to specific G-protein-coupled receptors and signal cellular responses, e.g., inflammation, vasodilation, blood pressure, pain etc. But never before it was suggested/shown that C_{20:5n-3} (and C_{22:6 n-3}) PUFAs, e.g., EPA (and DHA), are transformed into stable aromatic metabolites, DBPs, which elicit a large array of bioactivities in the producer organisms and also control the synthesis and metabolism of arachidonate-derived prostaglandins. The major beneficial effects attributed to EPA and DHA are now found to be largely contributed by DBPs and their aminoacyl conjugates and the dibenzo-α-pyrone-chromoproteins (DCPs). Because of the highly unstable nature of EPA and DHA, when administered, they are metabolized into a large array of uncontrolled products, several of which are systemically undesirable. By contrast, DBPs, because of their stability, perform the biological response modifier (BRM) functions in a directed and sustained way. Many of the biological effects of DBPs described in this invention, were earlier attributed to EPA and DHA,—the precursors of DBPs.

### BACKGROUND OF THE INVENTION

1. Field of the Invention

This invention relates to compositions of stable (aromatic) metabolites of docosahexaenoic acid (DHA) and eicosapentaenoic acid (EPA), produced by enzymatic and non-enzymatic autooxidations of the polyunsaturated fatty acids (PUFAs). These metabolites are identified to be oxygenated dibenzo-α-pyrones (DBPs). Biological functions of these metabolites as well as their conjugates in pharmaceutical, nutritional, veterinary formulations are described.

2. Description of the Related Art

Fish oils are rich in essential fatty acids, viz eicosapentaenoic acid (EPA, C_{20:5 n-3}) and docosahexaenoic acid (DHA, C_{22:6 n-3}). Both EPA and DHA fall into an even larger category of polyunsaturated fatty acids (PUFAs). Compared to saturated fats, PUFAs are more readily used for energy when ingested. Increasing the degree of unsaturation at a given carbon chain length increases the relative mobility of stored fat, making PUFAs more bioavailable (Storlien, L. H., Higgins, J. A., Thomas, T. C., et al. (2000). Diet composition and insulin action in animal models, Br J Nutr, 83, S85-S90). EPA and DHA come from the PUFA, alpha-linolenic acid (ALA, C_{18:3 n-3}) and are classified as omega-3 fatty acids. The nomenclature of an omega-3 fatty acid indicates that the first carbon-carbon double bond occurs at the third carbon atom from the methyl end of the molecule. Through a series of enzymatic reactions, the 18:3 PUFA is converted first to EPA and then finally to DHA. Both EPA and DHA are deemed conditionally essential as the body can synthesize them from ALA. However, while consumption of ALA can lead to significant increases in tissue EPA, it does not do so for DHA (Mantzioris, E., Cleland, L. G., Gibson, R. A., et al. (2000). Biochemical effects of a diet containing foods enriched with n-3 fatty acids, Am J Clin Nutr, 72, 42-48). There are several circumstances where the requirements for DHA greatly exceed the rate of synthesis, making supplementation necessary.

This application is related to U.S. Pat. Nos. 6,440,436 B1 and 6,558,712 B1, U.S. patent application Ser. No. 10/799,104 filed Mar. 12, 2004 entitled “Oxygenated Dibenzo-α-Pyrone Chromoproteins” and U.S. patent application Ser. No. 10/824,271 filed Apr. 14, 2004, entitled “Oxygenated Dibenzo-α-Pyrone Chromoproteins”, by the same inventor, all of which are incorporated by reference herein.

Natural Occurrence of EPA and DHA and the Evolutionary Sequence in the Genesis of DBPs

Members of the phylum Labyrinthulomycota (Lb) (Kingdom, Stramenopile), called marine slime molds [protistans,—a branch-point between plant (phyta) and animal (metazoa)], are parasitic or saprotrophic on marine invertebrates, particularly mollusks (to which Ammonites, the precursors of shilajit belongs), aquatic plants and organic debris. The families of Lb include Thraustochytriaceae (Th). Th comprises nine genera and thirty species. Schizochytrium (Sz) species, an important member of the family Th, can grow on all types of mollusks, including shells.

Sz is used as a commercially produced source of Omega-3-fatty acids (polyunsaturated fatty acids (PUFAs)) for enrichment of rotifers (Brachionus sp.) and brine shrimp (Artemia nauplii) with PUFAs, prior to feeding them to fish, as essential nutrients, a process common in aquaculture industry.

Sz species, a heterotrophic micro alga, is rich in n-3 (=Omega-3) and n-6 (=Omega-6) series of polyunsaturated fatty acids, namely, C_{22:6 n-3} (DHA) and C_{22:5 n-6} (docosapentaenoic acid, DPA), respectively. The spray-dried cells of Sz are very effective in enriching rotifers and brine shrimp in both n-3 and n-6 PUFAs. The brine shrimp and rotifers are capable of readily retroconverting DHA to EPA, and DPA to arachidonate (Scheme-I), usually through the process of β-oxidation, a process occurring in the mitochondria of metazoans. EPA and arachidonate compete for cycloxygenase for their transformation into DBPs and prostaglandins, respectively (Scheme-I). Hence, DBPs play a very significant role in the systemic formation and equilibrium of prostaglandins. These stable aromatic compounds (DBPs) prevent both unbridled production of the unstable prostaglandins and their rapid transformation into systemically adverse metabolites e.g., leukotrienes and thromboxanes.

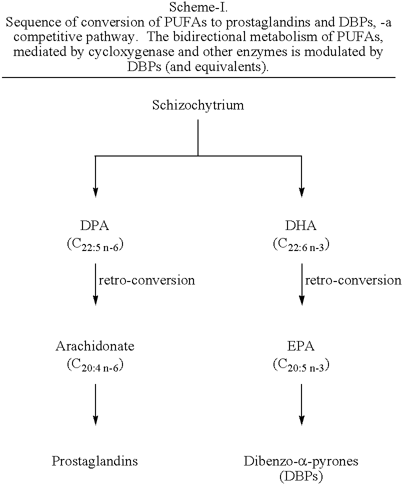

EPA and DHA compete with arachidonic acid (AA) for the enzyme cycloxygenase. EPA is converted by platelet cyclo-oxygenase to thromboxane A3 (TXA3), which is only a very weak vasoconstrictor, unlike thromboxane A2 (TXA2), which is formed by the action of cyclo-oxygenase on AA and is a strong vasoconstrictor. However, prostacyclin I3 (PGI3), formed from EPA in the endothelium, is as potent a vasodilator and inhibitor of platelet aggregation as is prostacyclin 12 (PGI2) formed from AA. The net effect, therefore, of an increased dietary EPA:AA ratio is relative vasodilation and platelet aggregation inhibition (Singleton, C. B., Walker, B. D., Cambell, T. J. (2000). N-3 polyunsaturated fatty acids and cardiac mortality, Aust N Z J Med, 30, 246-251). EPA yields the 5-series of leukotrienes, which are only weakly chemotactic. A relative reduction in chemotaxis might be expected to be antiatherogenic. Fish oil decreases both very low density lipoproteins (VLDLs) and triglycerides due to inhibition of hepatic triglyceride synthesis. Because VLDL is a precursor to LDL, a reduction in LDL cholesterol is seen in some patients with hypertriglyceridemia; however, fish oil does not appear to lower plasma cholesterol in subjects with hypercholesterolemia. (See Schectman, G., Kaul, S., Kissebah, A. H. (1989). Heterogeneity of low density lipoprotein responses to fish-oil supplementation in hypertriglyceridemic subjects. Arteriosclerosis, 9, 345-354; Wilt, T. J., Lofgren, R. P., Nichol, K. L., et al. (1989). Fish oil supplementation does not lower plasma cholesterol in men with hypercholesterolaemia. Results of a randomized, placebo-controlled crossover study, Ann Intern Med, 111, 900-905.)

Published clinical research has linked omega-3 acids consumption to health benefits in a number of areas. They include:

1. Coronary Heart Diseases
a. Thrombosis and homeostasis
b. Blood lipids
c. Atherosclerotic events
d. Hypertension
e. Ventricular fibrillation and cardiac arrhythmia
f. Restenosis after angioplasty
g. Insulin resistance syndrome
h. Cardiac transplant

2. Inflammatory Reactions
a. Inflammatory bowel disease
b. Rheumatoid arthritis
c. Skin disease
d. Lung disease
e. Other immune related conditions

3. Diabetes and Glucogen Storage Disease

4. Cancer
a. Breast cancer
b. Colorectal cancer

5. Other Diseases
a. Osteoporosis
b. Depression
c. Schizophrenia
d. Dyslexia, dyspraxia, and ADHA
e. Malaria
f. Renal disease
g. Peroxisomal disorders
h. Migraine

It is conceivable that these medicinal effects of EPA and DHA are mediated, at least partly, by the DBPs (and equivalents) formed systemically from the two PUFAs.

DHA and EPA have limited stability due to their susceptibility to autooxidation. The rate of DHA autooxidation is higher than that of EPA. Thirty-one volatile compounds were identified in ethyl ester (EE), and 23 volatile compounds in triacylglycerol (TG). (E)-2-pentenal, 2-(1-pentenyl) furan, and (E,E)-2,4-heptadienal were commonly detected as oxidized volatile compounds from TG and EE fish oil. These volatile oxidized compounds can form mainly from the oxidation of DHA and EPA, the main fatty acids of the oil (Lee, H., Kizito, S. A., Weese, S. J., Craig-Schmidt, M. C., Lee, Y., Wei, C. I. and An, H. (2003). Analysis of Headspace Volatile and Oxidized Volatile Compounds in DHA-enriched Fish Oil on Accelerated Oxidative Storage, J. of Food Sci., Vol. 68, No. 7), thereby limiting their use. The most stable compounds identified, in the present invention, from the autooxidation of EPA and DHA are the oxygenated dibenzo-α-pyrones (DBPs). The DBPs elicit a large array of beneficial effects, in living organisms, more pronounced than those of EPA or DHA.

### SUMMARY OF THE INVENTION

The present invention relates to compositions of stable aromatic metabolites of docosahexaenoic acid (DHA) and eicosapentaenoic acid (EPA), and their beneficial uses in human and animal health care.

In one embodiment, the invention provides a composition of stable metabolites of docosahexaenoic acid (DHA) and eicosapentaenoic acid (EPA) comprising of oxygenated dibenzo-α-pyrones (DBPs) and their conjugates.

Another embodiment of the invention includes oxygenated dibenzo-α-pyrones of formula (I) 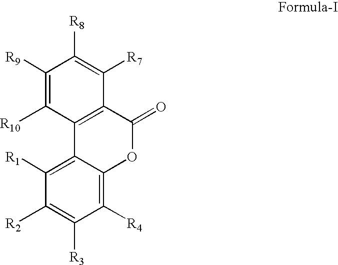
Wherein:
R₃ is selected from the group consisting of OH, O-acyl, O-aminoacyl, phosphocreatine;
R₈ is selected from the group consisting of H, OH, O-acyl, O-aminoacyl, phosphocreatine groups;
R₁, R₂, R₇, R₁₀ are independently selected from the group consisting of H, OH, O-acyl, O-aminoacyl, and fatty acyl groups;
R₉ is independently selected from the group consisting of H, OH, O-acyl, O-aminoacyl, fatty acyl groups, and 3,8-dihydroxy dibenzo-α-pyrone (DBP) groups;
O-acyl groups are selected from saturated and unsaturated fatty acids having carbon chain lengths of about C₁₄ to C₂₄; and
O-aminoacyl groups are selected from methionine, arginine, glycine, alanine, threonine, serine, proline, and hydroxyproline.

Another embodiment of the invention provides a pharmaceutical, veterinary or nutritional formulation comprising of DBPs or their conjugates present in an amount of about 0.05% to about 50% by weight.

Another embodiment of the invention provides a pharmaceutical formulation comprising DBPs or their conjugates wherein the pharmaceutical formulation is in the form of a tablet, syrup, elixir or capsule.

Another embodiment of the invention provides a nutritional formulation comprising DBPs or their conjugates wherein the nutritional formulation contains about 0.5% to about 30% by weight.

Another embodiment of the invention provides a veterinary formulation comprising DBPs or their conjugates wherein the veterinary formulation contains about 0.5% to about 30% by weight.

### BRIEF DESCRIPTION OF THE DRAWINGS

FIG. 1 shows the transformation of EPA to DBP in the absence and presence of catalytic amounts of FeSO₄.

FIGS. 2A and 2B show oral administration of EPA (cis-5,8,11,14,17-Eicosapentaenoic acid) to rat and tracking the blood level of DBPs by HPLC.

FIGS. 3A and 3B show HPLC-PDA spectra of two DBP fractions found in human blood plasma (upper curve) and in fossil of Trilobita (ca. 500 mybp) (lower curve).

FIGS. 4A-4D show Oral administration of DBPs [200 mg/kg, plasma (a) and blood cells (b); 300 mg/Kg, plasma (c) and blood cells (d)] to rats and tracking DBPs in the plasma and blood cells at different time intervals.

### DETAILED DESCRIPTION OF THE INVENTION

Interrelationship of DHA, EPA and the Oxygenated dibenzo-α-pyrones (DBPs)

An intimate relationship of the DBPs and the lipid fractions of the invertebrate fossils and of shilajit was discerned. DBPs were found in the organs and tissues of a large number and variety of land and marine animals. Two DBPs (str. 1 and 2, Scheme-II) were found in the renal caliculi of sheep; scent glands of Canadian beaver; feces of Ladakhian mouse and in the haemolymph of termites (Lederer, E. (1946). Castoreum pigment, Nature, 157, 231-232; and Lederer, E. (1949). Chemistry and biochemistry of some mammalian secretions and excretions, J. Chem. Soc. 2115-2119; Carroll, H. T. and Bennetts, H. W. (1956). Diseases of sheep in Western and Southern Australia, J. Dep. Agric. W. Aust., 5, 421-425; Pope, G. S. (1964). Occurrence of urolithins-A and -B in sheep, Biochem. J. 93, 474-477; Moore, B. P. (1964). The chemistry of nasutins, Aust. J. Chem. 17, 901-907. Interestingly, the contents of DBPs (str. 1 and 2, Scheme-II) were found to be appreciably higher in the sperm membranes, which are known to be rich source of both PUFAs and prostaglandins. Samples from a number of animals, viz, goat, ram and bull, were studied for the purpose (see experimental).

Consideration of the non-enzymatic chemical transformations of PUFA, e.g., EPA and DHA, calls to mind the unbridled autooxidation resulting in a host of metabolites including dicarboxylic acids (str. 3, Scheme-II) and their lactones, some of which were consistently present in the marine fossils and in shilajit. Another class of products, resulting from Diels-Alder-type reaction of PUFA, would produce unsaturated cyclic compounds and also phenolic compounds. The reaction may take place at ordinary temperature, particularly when PUFAs are present in free forms, in polar solvents, at slightly acidic pH. In fact, such a pathway of arachidonate (C_{20:4}n-6) transformation, involving oxidative free radical reaction was already reported. The reaction yielded a novel series of bioactive compounds termed isoprostanes (Morrow, J. D., Hill, K. E., Burk, R. F., Nammour, T. M., Badr, K. and Roberts, L. J. (1990). Prostaglandin F-2 like compounds by a non-cyclooxygenase free radical catalyzed mechanism, Proc. Nat. Acad. Sci. USA, 87, 9383-9390). Under a wide variety of marine and stratigraphic conditions, a broad range of cyclic compounds including the DBPs (Scheme-III) might conceivably be produced from EPA and DHA. The presence of transition metal ions would facilitate such reactions. In order to test this possibility, the following experiments were conducted.

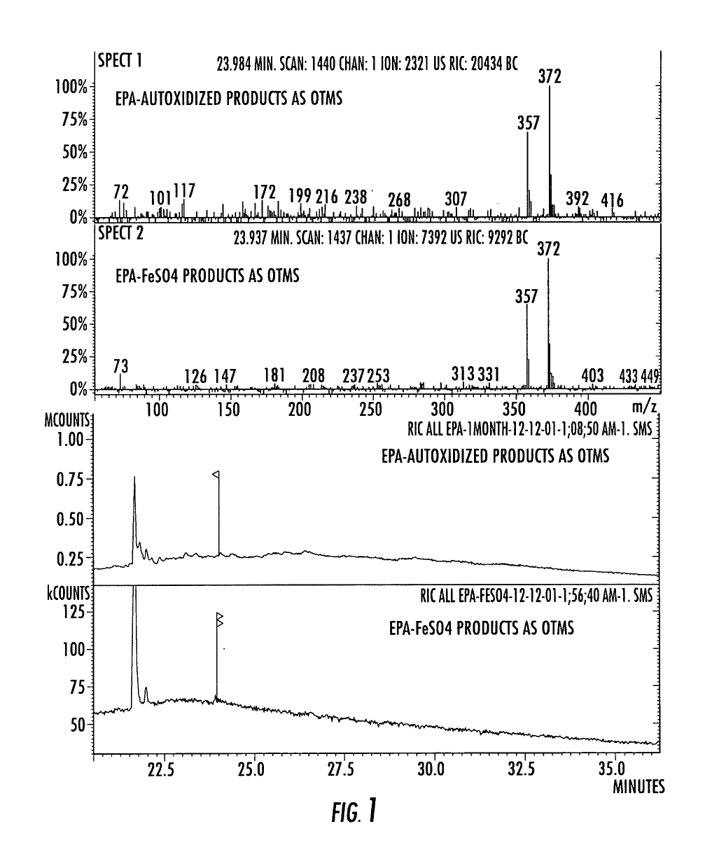

In an in vitro experiment, EPA (eicosapentaenoic acid) on autooxidation produced a mixture of DBPs and benzoic acid. The compound (EPA) did not exhibit the presence of any detectable amount of DBP at the onset of the reaction. The products were analyzed by GC-MS (Gas-Chromatography-Mass Spectrometry), as the TMS derivatives. The yields of the DBPs and benzoic acid were appreciably increased in presence of catalytic amounts of FeSO₄ (FIG. 1).

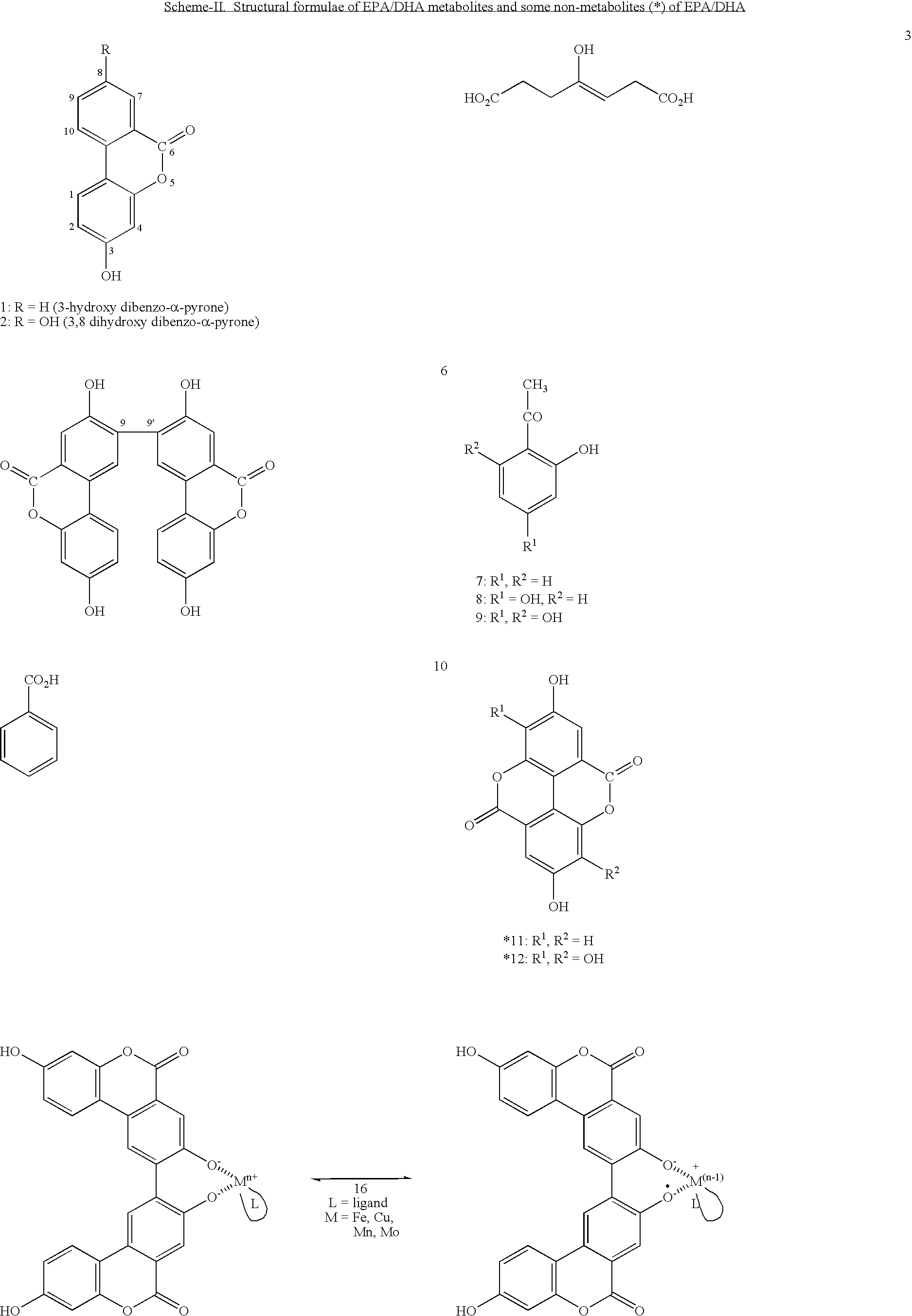

Since in the event of systemic deficiency of EPA, in living animal organisms, DHA is converted into EPA (Nordoy, A. (1991). Is there a rational use for n-3 fatty acids (fish oil) in clinical medicine? Drugs, 42, 331-342), the autooxidation of DHA was also studied. DHA (5) was subjected to similar autooxidation in vitro, as meted to EPA. The formation and augmentation of DBPs (1,2,6) and hydroxyacetophenones (7-9) (Scheme-II) were monitored by GC-MS (as TMS derivatives) and HPLC of the products. The findings supported the postulates depicted in Scheme-III.

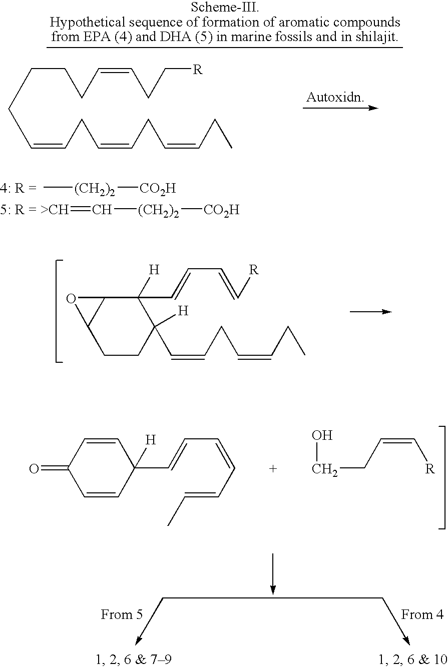

Many land animals were reported earlier to contain DBPs (and equivalents) in their different organs and organelles. It was to be determined if these DBPs were systematically produced from EPA/DHA. This hypothesis was tested by feeding EPA and DHA separately, to laboratory animals when augmentation of DBPs and benzoic acid (from EPA) in the blood samples of the treated animals was observed.

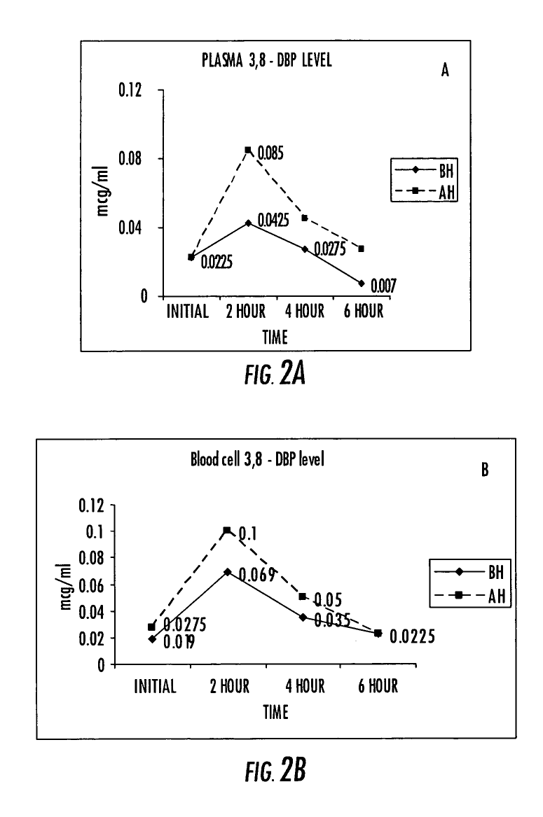

Oral administration of EPA to albino rats and tracking the blood level of DBPs by HPLC were conducted. EPA (25 mg in 0.5 ml propyleneglycol) was orally administered to each rat and the blood (1 ml) was withdrawn just before and after 2, 4, 6 hours of administration of this DBP-precursor (EPA). Cells and plasma were separated by centrifugation and extracted separately with methanol before (BH) and after acidic (HCl) hydrolysis (AH). These extracts were subjected to HPLC, when DBPs (3-hydroxy- and 3,8-dihydroxydibenzo-α-pyrones) so formed were tracked and estimated. FIGS. 2A and 2B show the turnover of EPA into 3,8-dihydroxydibenzo-α-pyrone. That DBP was quickly converted into the conjugates was revealed from the higher concentrations of DBPs in the plasma and cells after the acidic hydrolysis. The base level of DBP was maintained even after 6 hours (determined up to 72 hours, not shown in the FIGS. 2A and 2B).

The findings from the in vitro and in vivo experiments strongly support the postulate that the unique chemical constituents, viz. DBPs, of shilajit and marine fossils had their origin, at least partly, in EPA and DHA (and equivalents) (Scheme-II). These compounds were found completely absent in plants and microorganisms. The biogenetic origin of the polyunsaturated fatty acids, the precursors of EPA and DHA, can be traced back to Schizochytrium and related species (Kingdom Stramenopila). In the placement among Eukaryotes, Stramenopiles were grouped with animal phyla, and other protists. The process of retroconversion, by α-oxidation, of DHA is known to occur in the peroxisomes and mitochondria of rotifers and Artemia sp. It involves two reactions: (1) the DHA (C_{22:6 n-3}) or DPA (C_{22:5 n-6}) loses its double bond in position 4, a reaction involving the enzyme 4-enol-CoA reductase, while the carbon chain length remains unaltered; and (2) chain shortening to C_{20:5 n-3} or to C_{20:4 n-6}, respectively, then takes place (Scheme-I). The exclusivity of occurrence of DBPs in the animal kingdom (and not in plants) is thus conceivable.

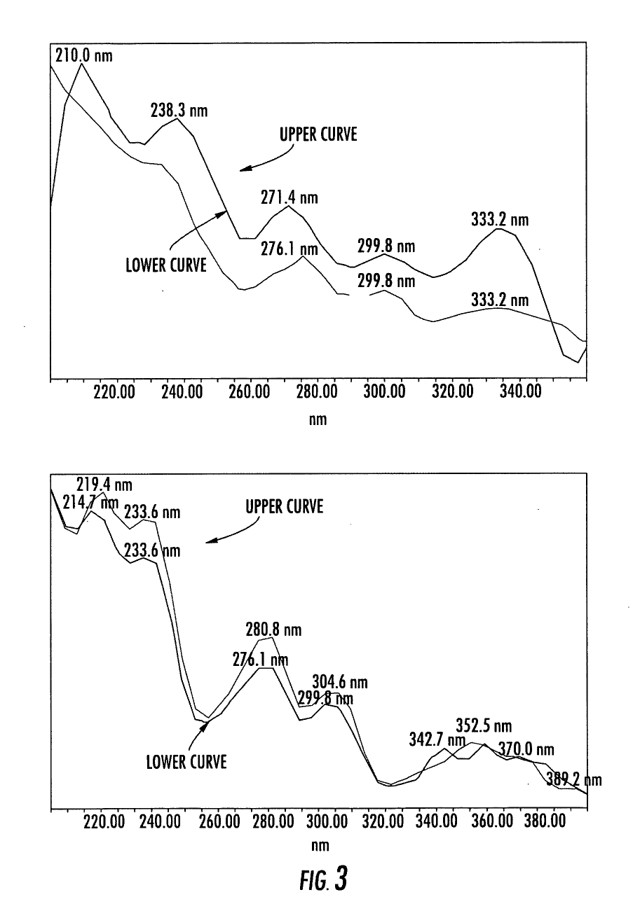

The occurrence of the two DBPs (1 and 2, Scheme-II) was subsequently established in many other living animals, e.g., in zoo-planktons, silk-pupa, shrimp, crabs, octopus and in the blood plasma of humans. In this context, it is significant that two HPLC eluates comprising the DBPs, from human blood plasma, showed superimposable UV spectral patterns, when compared with the DBP-fraction—extracts from fossil of Trilobite (Arthropoda, α-500mybp) (FIGS. 3A and 3B).

Plants are prolific producers of low and high molecular weight chemical compounds known as the secondary metabolites. Yet, when over forty different plant species, belonging to 30 genera of 18 families, growing in the shilajit-bearing rocks of the Kumaon region, were analyzed, none of them was found to contain DBPs (which are the essential building units of shilajit bioactives).

The unique oxygenation patterns (3- and 3,8-) of the shilajit—DBPs and the absence of any alkyl (or equivalent) substituent in the DBP-nuclei are the hallmarks of their distinct characters. These patterns differentiate them from the other α-pyrone phenolics of plant and microbial origin (Ghosal, S. (1990). Chemistry of shilajit, an immunomodulatory Ayurvedic rasayan, Pure & Appl. Chem., 62, 1285-1288; Ghosal, S., Lal, J., Bhattacharya, S. K., et al., 1991. The need of formulation of shilajit by its isolated active constituents, Phytother. Res., 5, 211-216; Ghosal, S. (1992a). Shilajit: its origin and significance in living matter, Indian J. Indg. Med. 9, 1-3; Ghosal, S. (1992b). The saga of shiljait, Proceedings of 2^{nd} Indo-Korean Symposium on natural products, Seoul, Korea, (Plenary lecture), pp. 1-12; Ghosal, S. (1993). Shilajit: Its origin and vital significance, In: Traditional Medicine, ed. by B. Mukherjee, Oxford—IBH, New Delhi, p. 308-319).

Thus, the unsymmetrical oxygenation pattern (str. 1, Scheme-II), in the absence of a C₈—OH, would rule out its formation from the symmetrical phenolic coupling of m-hydroxybenzoic acids. Again, the dilactone (11, Scheme-II), resulting from the symmetrical coupling of 3-hydroxy or 3,5-dihydroxybenzoic acids, was completely absent in shilajit. Likewise, another product (12, Scheme-II), that would result from the hypothetical coupling of gallic acid was also absent in shilajit. These facts would mean that straightforward phenolic coupling of the naturally occurring phenolic (mono-, di-, trihydroxy-) acids were not involved in the genesis of DBPs. The absence of a methyl substituent (or its equivalent, e.g., —CH₂OH, —CHO or —CO₂H) at C₁-position of any of the DBPs, occurring in shilajit, would rule out the genesis of DBPs from fungi like the Alternaria sp. Alternaria sp. were found to produce C₁-methyl substituted dibenzo-α-pyrones, e.g., alternariol (and equivalents) (Raistrick, H., Stickings, C. E. and Thomas, R. (1953). Alternariol and alternariol monomethylether. Metabolic products of Alternaria tenuis, Biochem. J. 55, 421-425; Starratt, A. N. and White, G. A. (1968). Identification of some metabolites of Alternaria cucumerina (E. & E.) Ell., Phytochemistry, 7, 1883-1884). Exhaustive GC-MS analyses of silylated shilajit products were conducted to test the validity of these contentions. The findings validated the postulate that plants were not the sources of DBPs.

Another conceptual model considered for the genesis of DBPs was the condensation of prephenate (bold line, Scheme-IV) and acetate malonate precursors. The intermediate (13, Scheme-IV) would lead to either 3,7-(14, Scheme-IV) or 3,9-dioxygenated (15, Scheme-IV) product. None of these compounds (14 or 15, Scheme-IV) were encountered in shilajit. Thus, all the plausible phytochemical sequences considered for the genesis of shilajit-DBPs have failed to provide the proof of existence of DBPs in plants. By contrast, the origin of the DBPs in animals has been further supported by the observations that these compounds (1 and 2, Scheme-II) occur in the organ deposits and in secretions and excretions of a large number of animals and insects (but not in plants).

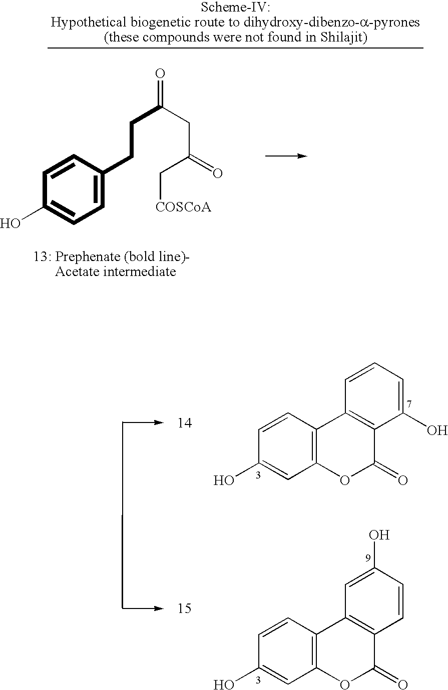

The special food habit of beaver, consisting of buds and barks of trees, was believed to be responsible for the deposit of DBPs in their digestive organ (Lederer, E. (1946). Castoreum pigment, Nature, 157, 231-232; Lederer, E. (1949). Chemistry and biochemistry of some mammalian secretions and excretions, J. Chem. Soc. 2115-2119). Lederer further pointed out that the two DBPs (1 and 2, Scheme-II) had a close structural similarity to ellagic acid (12, Scheme-II). However, no evidence was adduced in support of the postulate that systemic reduction (removal of hydroxyl groups) and removal of one lactone ring might lead to (1, Scheme-II) and (2, Scheme-II). The complete absence of (11, Scheme-II) and (12, Scheme-II) in shilajit, as established by comprehensive HPLC and GC-MS analysis (of silyl derivatives), using authentic markers, ruled out the possibility of formation of DBPs (1 and 2, Scheme-II) from the gallo-ellagi tannoids (Ghosal, S., Mukhopadhyay, B. and Bhattacharya, S. K. (2001). Shilajit: a rasayan of Indian Traditional Medicine, Molecular Aspects of Asian Medicine, Vol. 1, PJD, Westbury, N.Y., 425-444; Ghosal, S. (2002a). Process for preparing purified Shilajit, composition from native shilajit, U.S. Pat. No. 6,440,436 B1; Ghosal, S. (2002b). Delivery system of pharmaceutical, nutritional and cosmetic ingredients. U.S. Pat. No. 6,558,712 B1. However, although gallo-ellagi tannoids are not the precursors of DBPs, systemic administration of small gallo-tannoids do increase the synthesis of DBPs, presumably, via modulation of the EPA/DHA-cycloxygenase pathway.

Another significant observation regarding the DBPs has been their primordial nature of existence (Ghosal, S. (1997). Ayurvedic maharasas, the repository of primordial organic chemistry, J. Indian Chem. Soc. 74, 930-936 (hereinafter referred to as “Ghosal 1997”). These compounds (1,2,6, Scheme-II) were found present in the inner core of terminal morane (till) and boulders of Gangotri glacier (Ghosal 1997). The core of the siliceous bodies was found to be intimately mixed with a large variety of organic compounds, e.g., phenolic and aromatic carboxylic acids, amino acids, lipids and sugars. Optical microscopy of thin sections of the pebbles revealed light-brown to blackish-brown streaks of organic deposits, distributed in laminations parallel to the bedding planes. The inner surface distribution and complexation of the organic compounds indicated their original sedimentary deposition characteristics that had happened prior to the compaction of inner siliceous matrix. The groundmass of the rock-till was greyish in color. X-ray powder data showed the presence of quartz, felspar, and pyrites in combination with clay particles. Scanning electron microscopy (SEM) of the particles revealed spheroid and elliptical voids in the inner matrices in which the organic compounds were found embedded. Determinations of the concentrations of K and the Rb/Sr ratio suggested the age of the rock matrix to be well over 1 million years.

In a typical experimental study, the organic materials were partially dissociated from the organo-mineral laminar surfaces by repeated trituration with organic solvents of graded polarity, e.g., hexane, chloroform, ethyl acetate, methanol and n-butanol. HPTLC, HPLC and GC-MS analysis (of the silyl derivatives) of the organic solvent extractives showed the presence of a large number and variety of organic compounds, all of which were earlier found in shilajit (Ghosal, S., Lal, J., Bhattacharya, S. K., et al., 1991. The need of formulation of shilajit by its isolated active constituents, Phytother. Res., 5, 211-216; Ghosal, S. (1993). Shilajit: Its origin and vital significance, In: Traditional Medicine, ed. by B. Mukherjee, Oxford—IBH, New Delhi, p. 308-319. An inner section of the pebble was dipped in hydrofluoric acid, to dissolve the contained minerals; the acid-treated insoluble material was washed with water, dried and powdered. A portion of the powdered material was suspended in water and the aqueous suspension was triturated with Dowex-50 (H+)-resin. The effluent was extracted successively with ethyl acetate and n-butanol. The residues from the organic solvent extracts were analyzed by (i) HPTLC and HPLC, using DBP-markers (1,2,6, Scheme-II); and (ii) GC-MS of the corresponding silyl derivatives. These studies established the presence of DBPs and their oligomeric equivalents in the rock pebbles of the Gangotri glacier. The marine origin of shilajit and its major bioactives, the DBPs and conjugates, is thus projected.

Thus, plants do not seem to elaborate DBPs, neither do bacteria nor fungi. By contrast; organisms in which DBPs occur quite commonly are the animals (as mentioned before). However, several factors render the possibility of formation of DBPs and shilajit, to any appreciable extent, from land animals rather remote: (i) the low content of DBPs in land animals and, by contrast, the abundant reserves of shilajit humus; with high contents of DBPs in (ii) shilajit-bearing steep rocks not negotiable by land animals; and (iii) ecological variations in shilajit-bearing rocks worldwide would not permit consideration of any particular land animal as the source of DBPs. Also, the contents of EPA and DHA are much higher in marine animals than in land animals. Hence, marine animals are regarded as the major sources of DBPs and equivalents. The inventor has earlier shown that marine invertebrates (fossils and dead animals) constitute the major source material of shilajit (U.S. patent application Ser. No. 10/799,104 filed Mar. 12, 2004 entitled “Oxygenated Dibenzo-α-Pyrone Chromoproteins” and U.S. patent application Ser. No. 10/824,271 filed Apr. 14, 2004, entitled “Oxygenated Dibenzo-α-Pyrone Chromoproteins”, by the same inventor).

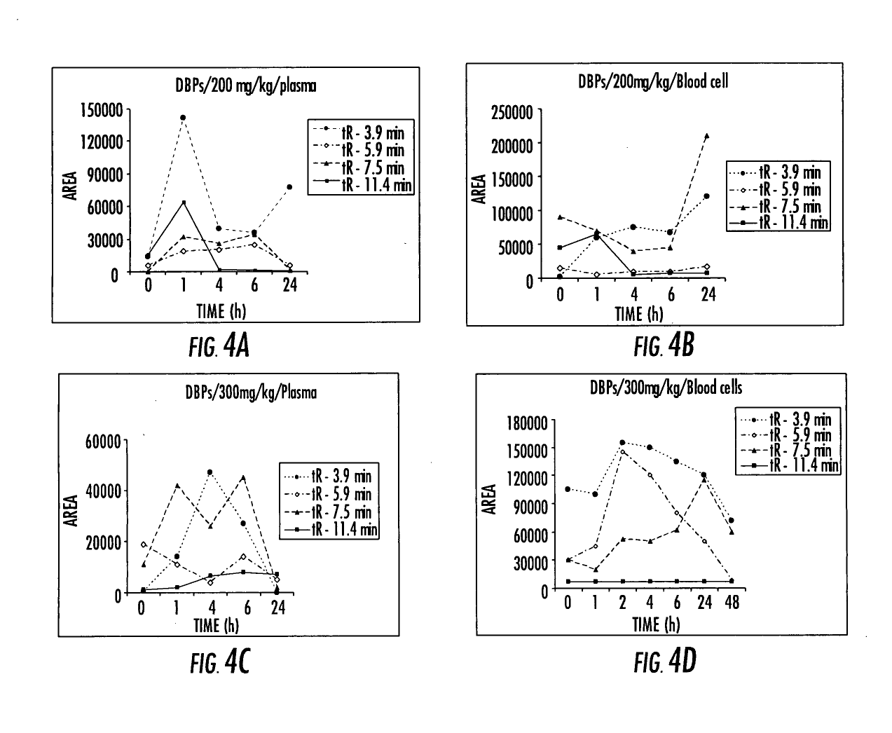

The biochemical significance of DBPs (1 and 2, Scheme-II) was revealed by their oral administration to laboratory animals when they were converted dynamically into the corresponding amino-acyl conjugates, e.g., 3-O-acylglycinoyl, 3-O-acylarginoyl, 3,8-di-O-acylphosphocreatinoyl and 3,8-di-O-acylpeptido-conjugates (FIGS. 4A-4D.) The systemic transformation of 3-hydroxy- and 3,8-dihydroxydibenzo-α-pyrone into the aminoacyl conjugates, comprising glycine, arginine, phosphocreatine (and equivalents), as revealed from the subsequent acid hydrolysis and GC-MS analyses (as TMS derivatives) of the products (HPLC-t_{R}: 3.9, 5.9, 7.5 and 11.4 min.), suggest the significance of DBPs in systemic metabolism. Very similar conjugates were found to occur in dibenzo-α-pyrone chromoproteins (DCPs), isolated from shilajit and its precursors, -ammonites, corals and other invertebrates, and human blood (U.S. patent application Ser. No. 10/799,104 filed Mar. 12, 2004 entitled “Oxygenated Dibenzo-α-Pyrone Chromoproteins” and U.S. patent application Ser. No. 10/824,271 filed Apr. 14, 2004, entitled “Oxygenated Dibenzo-α-Pyrone Chromoproteins”). The above observations and the systemic assimilation and turnover of these DCP constituents, when DCPs were fed to rats through oral route (DCP patent application), suggest the role of these compounds in energy storage in living system.

Arginine phosphate plays an important role in the storage of energy in invertebrates; the same role is played by creatine produced from a combination of argininephosphate and glycine phosphate in vertebrates. Creatine phosphate and arginine phosphate are reserves of phosphates of high energetic potential and, hence, the name ‘phosphagens’ given to these compounds as shown below (Scheme-V):

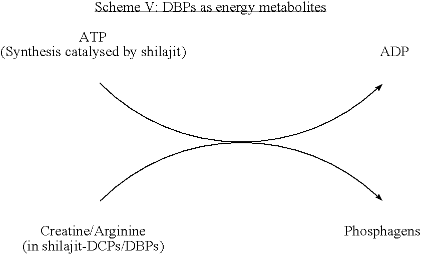

An energetic coupling represents the energy storage reaction when ATP is present in excess and, inversely, the formation of ATP by the reverse reaction when the cells need the ATP. Should we consider the biosynthesis and balance of DBP-phosphagen complexes in living organisms as the indices of their energy status, then in the event of dearth of these phosphagens, administration (p.o.) of DBPs (or their conjugates) would replenish them.

Biological Effects of DBPs

Oxygenated dibenzo-α-pyrones (DBPs, strs. 1, 2, 6 and equivalents, Scheme-II) are among the first group of natural tricyclic phenolic compounds of animal origin that appeared some 500-million-years before present time (MYBP) (FIGS. 3A and 3B). DBPs modulate the synthesis and systemic functions of one of the most potent hormones,—the eicosanoids. They maintain equilibrium in the “central nervous system (CNS)-immune-endocrine tripoidal system” in advanced aerobic organisms (animals and humans). The selected biological paradigms and effects thereof, as described in the experimental section under “Biological Effects”, would justify these postulates regarding the DBPs. These are:
a. Anti-ulcerogenic
b. Anti-inflammatory
c. Anti-stress agent
d. Modulator of arachidonic acid metabolism
e. Cognition enhancing and memory booster
f. Chronic stress reducer
g. Antioxidant
h. Anti-craving agent
i. Anti-anemic agent
   DBPs were found to be superior to DHA and EPA in the above tests.
   Pharmaceutical, Nutritional and Veterinary Formulations

The compositions herein may contain the inventive compound alone, or in combination with a pharmaceutically or nutritionally acceptable excipient, in dosage unit forms such as tablets, coated tablets, hard or soft gelatin capsules or syrups. These administrable forms can be prepared using known procedures, for example, by conventional mixing, granulating, tablet coating, dissolving or lyophilisation processes. Thus, pharmaceutical or nutritional or veterinary compositions for oral administration can be obtained by combining the active ingredient with solid carriers, optionally granulating the resulting mixture, and processing the mixture by granulation, if desired or necessary, after the addition of suitable excipients, to give tablets or coated tablet cores.

Suitable excipients are, in particular, fillers, such as sugars, for example, lactose, sucrose, mannitol or sorbitol; cellulose preparations and/or calcium phosphates, for example, tricalcium phosphate or calcium hydrogen phosphate; and binders, such as starches, for example, corn, wheat, rice or potato starch, gelatin, tragacanth, methyl cellulose and/or polyvinylpyrrolidone, and/or, if desired, disintegrants, such as the above mentioned starches, and also carboxymethyl starch, cross-linked polyvinylpyrrolidone, agar, alginic acid or a salt thereof such as sodium alginate, and/or flow regulators and lubricants, for example, silica, talc, stearic acid or salts thereof such as magnesium stearate or calcium stearate, and/or polyethylene glycol. Coated tablet cores can be provided with suitable coatings, which if appropriate are resistant to gastric juices, using, inter alia, concentrated sugar solutions which may contain gum arabic, talc, polyvinylpyrrolidone, polyethylene glycol and/or titanium dioxide, shellac solutions in suitable organic solvents or solvent mixtures or, for the preparation of coatings resistant to gastric juices, solutions of suitable cellulose preparations such as acetylcellulose phthalate or hydroxypropylmethylcellulose phthalate. Dyes or pigments can be added to the tablets or coated tablets, for example, to identify or indicate different doses of the active compound ingredient.

The orally administered vehicle in these formulations normally has no therapeutic activity and is nontoxic, but presents the active constituent to the body tissues in a form appropriate for absorption. Suitable absorption of the inventive compound normally will occur most rapidly and completely when the composition is presented as an aqueous solution. However, modification of the vehicle with water-miscible liquids or substitution with water-immiscible liquids can affect the rate of absorption. Preferably, the vehicle of greatest value for the present inventive composition is water that meets the USP specification for water for injection. Generally, water of suitable quality for compounding will be prepared either by distillation or reverse osmosis to meet these USP specifications. The appropriate specifications for such formulations are given in Remington: The Science and Practice of Pharmacy, 19th Ed. at p. 1526-1528. In preparing formulations, which are suitable for oral administration, one can use aqueous vehicles or carriers, water-miscible vehicles or carriers, or non-aqueous vehicles or carriers. Water-miscible vehicles or carriers are also useful in the formulation of the composition of this invention. The most important solvents in this group are ethyl alcohol, polyethylene glycol, and propylene glycol.

Another useful formulation is a reconstitutable composition which is a sterile solid packaged in a dry form. The reconstitutable dry solid is usually packaged in a sterile container with a butyl rubber closure to ensure the solid is kept at an optimal moisture range. A reconstitutable dry solid is formed by dry filling, spray drying, or freeze-drying methods. See Pharmaceutical Dosage Forms: Parenteral Medications, 1, p. 215-227.

Additional substances may be included in the compositions of this invention to improve or safeguard the quality of the composition. Thus, an added substance may affect solubility, provide for patient comfort, enhance the chemical stability, or protect preparation against the growth of microorganisms. The composition also may include an appropriate solubilizer, or substances which act as antioxidants, and a preservative to prevent the growth of microorganisms. These substances will be present in an amount that is appropriate for their function, and will not adversely affect the action of the composition. Appropriate antioxidants are found in Remington (p. 1529). Examples of suitable antimicrobial agents include thimerosal, benzethonium chloride, benzalkonium chloride, triclosan, methyl p-hydroxybenzoate, propyl p-hydroxybenzoate, and parabens.

Preferred pharmaceutical or nutritional formulations are those suitable for oral administration to warm-blooded animals.

Other pharmaceutical or nutritional preparations suitable for oral administration are hard gelatin capsules and also soft gelatin capsules made from gelatin and a plasticizer such as glycerol or sorbitol. Hard capsules may include the inventive compound in admixture with fillers such as lactose, binders such as starches, and/or lubricants such as talc or magnesium stearate, and if desired, stabilizers. In soft capsules, the inventive compound is preferably dissolved or suspended in a suitable liquid, such as fatty oil, paraffin oil or a liquid polyethylene glycol, to which a stabilizer can be added.

The following examples will serve to further typify the nature of the invention.

### EXAMPLE 1

### Chemical Synthesis of 3-hydroxydibenzo-α-pyrone

2-Bromobenzoic acid (5.8 grams), resorcinol (5.5 grams) and sodium hydroxide (2 grams) in water (25 ml) are heated under reflux for 10 minutes. After the addition of aqueous copper sulphate (5%, 10 ml), the mixture is refluxed again for 10 min. At the completion of the heating, 3-hydroxydibenzo-α-pyrone precipitated as a cream colored amorphous powder (8.7 grams). It was crystallized from ethyl acetate as micro-crystalline solid, m.p. 230-232° C.

### EXAMPLE 2

### Chemical Synthesis of 3,8-dihydroxydibenzo-α-pyrone

A mixture of 2-bromo-5-methoxybenzoic acid (5.6 grams), resorcinol (5.5 grams) and sodium hydroxide (2.2 grams) in water (25 ml) was heated under reflux for 30 minutes. After the addition of copper sulphate (5% aqueous solution, 10 ml), the mixture is refluxed again for 10 min when 3-hydroxy-8-methoxydibenzo-α-pyrone (3.7 grams) was precipitated as a straw colored powder. Crystallization from methanol and glacial acetic acid, in succession, afforded pale-yellow micro-crystals, m.p. 285-286° C. A suspension of this compound (2.18 grams) in a mixture of glacial acetic acid (120 ml) and azeotropic hydrobromic acid (60 ml) was heated under reflux for 11 hours. The starting material had dissolved within 2 hours and the desired product, 3,8-dihydroxydibenzo-α-pyrone (2), crystallized out after 6 hours as light yellow powder (1.9 grams). Recrystallization of the product from glacial acetic acid gave pale-yellow needles, m.p. 360-362° C. The purity of the products was determined by HPLC, and ¹H-NMR spectra.

### EXAMPLE 3

### Chemical Synthesis of 3,3′,8,8′-tetrahydroxy-9,9′-bis-dibenzo-α-pyrone (str. 6, Scheme-II),—the DBP-dimer

Methanolic solutions of 3,8-dihydroxydibenzo-α-pyrone (2) (102 mg) and phosphomolybdic acid (108 mg) were mixed and then adsorbed on silica gel (60-120 mesh, 1 gram). It was desiccated and the residue was charged on top of a chromatographic column (silica gel, 12 grams). The column was moistened with light petrol and kept overnight at room temperature (25° C.±5° C.). Elution of the column with ethyl acetate-toluene (10:90) separated (6) as a yellowish-orange layer. The solvent was evaporated and the residue, an amorphous yellowish-orange powder (41 mg), was collected. A further crop (7 mg) was obtained by eluting the column with aqueous-acetone. Thus, DBPs on autooxidation are converted into a yet stable bioactive product, the dimer (6, Scheme-II).

### EXAMPLE 4

### Metal Ion Chelating Property of DBP-Dimers

The ESR and UV-V is spectral characteristics of fulvic acids (FAs), from shilajit, of which the DBPs (and equivalents) are the major bioactives, suggested the presence of resonance-stabilized semiquinone-hemiquinone-containing condensed aromatic nuclei. The stability of these soft-spin (more bioactive) metallo-complex free radicals was augmented by metal ion complexation and chelation. Aqueous methanolic solutions of (6, Scheme-II), when separately treated with FeCl₃, Cu(OAc)₂ and Zn(OAc)₂ in 4-6:1 mM proportions readily formed such metal ion complexes (16, Scheme-II) as differently colored free-flowing powder. These metal ions bound and protected by tetra-(planar) and hexa-(octahedral) coordination offer resistance to invasive/noxious stimuli, e.g., oxygen and nitrogen free radicals and microbial enzymes. Hence, application of DBPs, systemically produce a cascade of biological effects as such and via their dimers (hemiquinone and semiquinone and other equivalents),—effects not elicited by their original precursors, viz. EPA and DHA.

### EXAMPLE 5

### Chemical Synthesis of 3-O-glycinoyldibenzo-α-pyrone

Condensation of 3-hydroxydibenzo-α-pyrone with tert-butyloxycarbonyl (BOC) glycine (Aldrich), in presence of dicyclohexylcarbodiimide (DCC), produced 3-O-(BOC)-glycinoyldibenzo-α-pyrone. Deblocking of BOC, from the product, with trifluoroacetic acid, afforded 3-O-glycinoyldibenzo-α-pyrone (the ubiquitous 3-hydroxydibenzo-α-pyrone conjugate in shilajit-dibenzo-α-pyrone chromoproteins).

### EXAMPLE 6

### Occurrence of DBPs in Laboratory Animals

Blood samples (2.5 ml) were collected from albino rats (200-220 grams, b.w.) by retro-orbital puncture, in heparinized tubes and centrifuged (3000 rpm) for 5 min. The supernatant (plasma, 0.4 ml) was collected and extracted with methanol (5 ml×3), at 60° C. by sonication for 10 min each. The combined methanolic extract was filtered and evaporated in vacuo. The residue so obtained was subjected to HPLC and GC-MS (as TMS derivatives) analyses. The presence of both 3-hydroxy-(str. 1, Scheme-II) and 3,8-dihydroxy-dibenzo-α-pyrone (str. 2, Scheme-II) was detected. Thus, the two DBPs are the normal metabolites of the albino rats. Their normal concentrations (control value) in the experimental rat blood were estimated at 0.170±0.052 μg/ml (str. 1, Scheme-II) and 0.100±0.023 μg/ml (str. 2, Scheme-II).

### EXAMPLE 7

### Augmentation of DBPs by EPA Treatment to Albino Rats

In the EPA treatment experiment to the above animals, EPA (25 mg, in propylene glycol, 0.5 ml) per rat was fed through the oral route. The control rats were fed only the propylene glycol. Blood was then collected from the EPA treated and control rats and processed as before. The changes in the amounts of DBP, at regular time intervals, in the control and the treated rats were noted by HPLC and GC-MS analysis. The progressive increase in the amounts of 3,8-(OH)2-DBP (2), and then decrease towards the control value were noted. However, the level of 2 was higher than that of the control even after 24 h of EPA treatment. After 72 h, it came down to control level.

The augmentation of benzoic acid (10, Scheme-II), after the EPA treatment was concomitantly observed (0.37±0.02 μg/ml pretreatment to 0.51±0.11 μg/ml post-EPA treatment). Similar transformations (formation of DBPs and hydroxyacetophenones, 7-9, Scheme-II) were observed in vivo after DHA treatment to albino rats. The augmentation of 3,8-dihydroxy-dibenzo-α-pyrone was maximum at 2 hours and the increase was about 30±12% over the control value after the DHA treatment.

### EXAMPLE 8

### Isolation of DBPs from Goat Sperm Membrane

In a typical experiment, goat sperm membrane was ruptured by osmotic shock and then ultracentrifuged in presence of ficcol. The membrane thus separated was taken in an aqueous buffer (pH 7.2) and centrifuged (6000 rpm) for 15 min. The resultant pellet was dried in vacuum and then extracted with ethylacetate, by magnetic stirring for 2 h under N2 cloud. The ethylacetate extract was divided into two parts. One part was subjected to HPLC (using solvent-D) and GC-MS analyses (as TMS derivatives) for free DBPs. The other part was saponified with 5% methanolic-KOH, under reflux for 4 h, under N2 atmosphere. The product was worked up in the usual way for saponified and non-saponified compounds. The saponified fraction, comprising fatty acids and phenolic compounds, was extracted with diethylether. The residue from the ether extract was subjected to HPLC and GC-MS analyses as before. The DBPs (1 and 2, Scheme-II), obtained and quantitated from this fraction were found to be present in the form of acylated conjugates (Formula-I). The fatty acids liberated were largely saturated; palmitic and stearic being major components. Traces of PUFAs (with 4 to 6 unsaturations) were also detected. The amounts of free and conjugated DBPs in goat sperm membrane were estimated at 0.551 μg/mg and 2.710 μg/mg sperm membrane, respectively. In goat milk, the amounts of these DBPs were, respectively 0.042 μg/g and 0.073 μg/g milk.

### EXAMPLE 9

### Transformation of EPA and DHA into DBPs

Eicosapentaenoic acid (EPA, 10.4 mg, Aldrich, Mlw, USA) was taken in methanol (5 ml), and the mixture was kept at ordinary temperature (25±2° C.) for 7 days. EPA did not exhibit the presence of any detectable amount of DBPs (1 and 2, Scheme-II) at the onset of the reaction (beginning of day-1). After autooxidation for 7 days, the products were subjected to GC-MS analysis, as the trimethylsilyl (TMS) derivatives. A small portion of the transformed product of EPA (ca. 3 mg) was dissolved in chloroform-methanol (2:1, 5 ml). An aliquot (10 μl) of this solution was treated with N,O-bis (trimethylsilyl)-trifluoroacetamide (Wako) at 60° C. for 1 hour. A portion of the silyl derivatives was injected into the GC-MS assembly. The presence of 3,8-dihydroxy-dibenzo-α-pyrone and benzoic acid as TMS derivatives, in the mixture was detected (FIG. 1). Analysis of the product on day-2 showed the presence of 3-hydroxy-dibenzo-α-pyrone (C-13) and benzoic acid (C-7) in the mixture. Among the 20-C units of EPA, DBPs comprise 13-carbons and the remaining 7-carbons constitute benzoic acid. The yield of DBPs was appreciably increased (FIG. 1) when catalytic amount of ferrous sulphate (0.1 mg) was added to the autooxidation mixture.

The autooxidation of DHA was also studied similarly, when both 3-hydroxy- and 3,8-dihydroxy-dibenzo-α-pyrones (1 and 2, Scheme-II) were detected in the transformed products (monitored on day-2 to day-7). The remaining 9-carbons (C-22-C-13) of DHA, constituted hydroxyacetophenones (strs. 7-9, Scheme-II), which were also detected in the autooxidation mixture as their trimethylsilyl derivatives by GC-MS analysis.

Biological Efects of DBPs

### EXAMPLE 10

### Anti-Ulcerogenic Effect

DBPs (1 and 2, Scheme-II) (1:1 w/w, 10 mg/Kg p.o./day×4 days), in association with their bioactive carriers, fulvic acids (10 mg/Kg, p.o.) (U.S. Pat. No. 6,558,712 B1) significantly reduced non-chronic stress-induced (noxious chemical-induced) ulcer index in pylorus ligated albino rats, compared to the vehicle control and the aspirin (ASP)-treated groups. DBPs (1 and 2, Scheme-II) per se had no adverse effect on the protein content in the gastric juice, compared to the vehicle control; but they reversed the adverse effect of aspirin (ASP). ASP, as such, caused a significant increase in the protein content without changing the carbohydrate contents of the gastric juice thereby producing considerable decrease in the carbohydrate/protein ratio. Mixture of DBPs (1:1, 1 and 2, Scheme-II), on'the other hand, increased the contents of individual and the total carbohydrates and also the total carbohydrate/protein ratio in the gastric juice. The ratio of the total carbohydrate/protein was taken as the index of the mucin activity. The potent mucin activity of the DBPs suggest significant anti-ulcerogenic action. Additionally, while ASP caused an appreciable increase in the contents of DNA and protein in the gastric juice by shredding of cells, DBPs decreased their (DNA and protein) concentrations in the gastric juice.

Another essential criterion of determining the status of mucosal resistance/barrier is the state of mucus secretion. DBPs increase not only the mucosal cellular mucus, but also secrete more dissolved mucus in the gastric juice as evidenced by their effects on gastric juice carbohydrates and on the increased carbohydrate/protein ratio. This, along with the observed increase in mucosal stability by DBPs, suggests that DBP-induced changes in the mucosa assist the mucus to resist the damaging effects of noxious stimuli (e.g., oxidative free radicals and loose metal ions) and ulcerogens. EPA (10 mg/ml) and DHA (10 mg/ml), showed only weak anti-ulcerogenic effects in the above test. DHA in very high doses (200 mg/ml/day×4 days), in association with fulvic acid (10 mg/Kg), elicited similar anti-ulcer activity comparable to the DBPs.

### EXAMPLE 11

### Anti-Inflammatory Effect of DBPs

Mast cells are the major source of mediators of allergy and anaphylaxis. The effect of DBPs (1 and 2, Scheme-II, 1:1 mixture) was studied in relation to the degranulation and disruption of mast cells against a large array of noxious stimuli, e.g., antigen-induced and compound 48/80 (Sigma, St. Louis)-induced degranulation of mast cells. Additionally, the spasmogenic response of sensitized guinea-pig ileum, in presence and absence of DBPs, was studied. The contraction of guinea-pig ileum is associated with an explosive degranulation of mast cells and the action is responsible for the release of histamine. DBPs provided significant protection to antigen-induced degranulation of sensitized mast cells, markedly inhibited the antigen-induced spasm of sensitized guinea-pig ileum, and prevented mast cell disruption induced by compound 48/80. These observations justify the use of shilajit in the treatment of allergic disorders in Ayurvedic medicine, and locate, at least partly, the bioactivities of shilajit to DBPs.

### EXAMPLE 12

### Anti-Stress Effect of DBPs

DBPs (1 and 2, Scheme-II, 1:1 mixture, 50 mg/Kg, p.o./day×4 days), not only significantly reduced the severity of stress-induced (forced swimming stress ulcers in albino rats), they exhibited a pronounced anti-stress effect in mice. Rodents when forced to swim in a restricted place, from which they cannot escape, become immobile after an initial period of vigorous activity. The observed immobility signified behavioral despair, resembling a state of mental depression. Behavioral depression is a common consequence of stress. The significant anti-stress effects of DBPs (1 and 2, Scheme-II), was assessed by the considerable reduction in the period of immobility in the test compound treated mice. The significant anti-stress effect of DBPs was manifested by the drastic reduction in the period of immobility, under stressed condition (total duration of immobility, 194±14 sec.), to 114±6 sec.; p<0.001, by DBP-treatment [(1 and 2, Scheme-II; 1:1 w/w, 50 mg/Kg, p.o. for 4 days]. Either of EPA or DHA, in these doses elicited a very weak anti-stress response (statistically insignificant activity).

### EXAMPLE 13

### Effect on Arachidonate Metabolism

The effects of DBPs on arachidonic acid (AA) metabolism were tested in isolated human neutrophils. DBPs significantly inhibited the biosynthesis of AA-lipoxygenase pathway products, e.g., leukotriene-B₄ (LTB₄) and 5-hydroxyeicosatetraenoic acid (5-HETE) at 50 μg/ml concentration of 1:1 mixture of 1 and 2, Scheme-II.

### EXAMPLE 14

### Effect of DBPs on Memory and Learning

The passive avoidance test, in old albino rats was employed (Ghosal, S., Lal, J., Bhattacharya, S. K., et al., 1991. The need of formulation of shilajit by its isolated active constituents, Phytother. Res., 5, 211-216). A 1:1 mixture of 1 and 2, Scheme-II, (10 mg/Kg b.w., p.o.,×7 days), in albino rats, showed augmentation of learning acquisition and memory retrieval in deficient recipients. Shilajit containing these bioactive agents (DBPs) has also been suggested to have potential in the treatment of Alzheimer's disease by scientific evaluations. Systemic applications of DBPs have modified acetylcholinesterase (ACHE) activity in different areas of the brain. Induced increase in cortical muscarinic acetylcholine receptor capacity explains, at least partly, the cognition enhancing and memory-improving effects of DBP-containing formulations in animals and humans.

In the learning acquisition paradigm, in the control group, the number of shocked and unshocked trials required to reach the criterion of 10 correct conditional responses, were 14.33 and 43.70, respectively. In the shocked trials, while DBPs exhibited marginal shortening in the number, EPA and DHA were practically without any beneficial effect. However, in the unshocked trials, significant shortening (p<0.01) was observed in case of DBPs, while DHA in higher doses only showed noticeable (p<0.05) shortening (Table 1).

TABLE 1
Effects of DBPs, EPA and DHA on active learning in rats

| | Dose (in | | Number of trials to reach criterion | |
|---|---|---|---|---|
| Group | mg/ml) | n | Shocked trials | Unshocked trials |
| Control | — | 8 | 14.33 ± 1.21 | 43.70 ± 1.60 |
| (distilled water) | | | | |
| DBPs (1 and 2, | 2.5 | 10 | 11.22 ± 1.39 | 28.02^{b} ± 1.33 |
| Scheme-II, 1:1 | 5.0 | 10 | 10.05 ± 1.10 | 27.17^{b} ± 1.07 |
| mixture) | | | | |
| EPA | 2.5 | 10 | 13.82 ± 1.04 | 40.11 ± 1.88 |
| | 5.0 | 10 | 12.11 ± 2.01 | 38.14 ± 1.92 |
| DHA | 2.5 | 8 | 12.55 ± 2.03 | 39.33 ± 2.04 |
| | 5.0 | 8 | 12.78 ± 1.83 | 35.21^{a} ± 1.79 |

Values are means ± SEM; levels of significance (p)
^{a}<0.05,
^{b}<0.01, in relation to control group (Student's t-test)
The test compounds were administered orally (p.o.) once daily 45 min before trial for 4 days. [Tested according to the procedure: Ghosal, S., Lal, J., Jaiswal, A. K. and Bhattacharya, S. K. (1993). Phytother. Res., 7, 29-34.]

### EXAMPLE 15

### Comparative Study of the Effects of DBPs EPA and DHA on Chronic Stress

A comparative study of DBPs (1, 2, Scheme-II; 1:1 mixture), EPA and DHA was carried out to determine their relative adaptogenic potency against chronic stress in albino rats. The study is also relevant in view of the projected links of EPA and DHA to mental development in children which is severely retarded by chronic stress.

Rats were randomly assigned to control or stress groups. Those assigned to the stress groups were subjected to 1 hour foot-shock, through a grid floor, every day for 14 days. The duration of each shock (2 mA) and the intervals between the shocks were randomly programmed between 3-5 seconds and 10-110 seconds, respectively, to make the stress unpredictable.

EPA (Aldrich), DHA (Sigma) and DBPs were separately suspended/dissolved in 0.3% carboxymethylcellulose (CMC) in distilled water and administered orally (p.o.) for 14 days, starting on day 1, 60 min. prior to electro-shock. Control animals received only the vehicle in either unstressed or the stressed rats for the same period in a volume of 2 ml/Kg, p.o. Estimations were conducted on day 14, one hour after the last stress procedure and two hours after the last test compound or vehicle was administered.

Chronic stress (CS) significantly increased the incidence, number and severity of gastric ulcers. The three test compounds had, albeit in different extent, dose-related anti-ulcerogenic effect. The efficacy was in the order: DBPs>DHA>EPA (Table 2).

CS caused marked depletion of adrenal gland ascorbic acid and corticosterone concentrations with concomitant increase in plasma corticosterone levels. These findings suggest that the stress protocol used in this study induced pronounced stress. The three test compounds (DBPs, DHA and EPA) reversed, to different extents, these stress-induced adverse effects in a dose-related manner (the stress-attenuating actions were in the order DBPs>DHA>EPA). They had no per se effect on the indices of stress investigated (Table 3).

TABLE 2
Effects of DBPs, EPA and DHA on CS-induced gastric ulceration in
albino rats

| Treatment | | | | |
|---|---|---|---|---|
| groups | | | | Severity of |
| (mg/Kg, p.o.) | n | Ulcer incidence (%) | No. of ulcers | ulcers |
| Chronic stress | 12 | 100 | 19.8 ± 3.0 | 32.4 ± 5.1 |
| (CS) | | | | |
| EPA₍₅₎ + CS | 10 | 70 | 16.5 ± 3.4 | 28.3 ± 7.7 |
| EPA₍₁₀₎ + CS | 10 | 60 | 14.3 ± 4.4 | 26.4 ± 6.2 |
| DHA₍₅₎ + CS | 10 | 70 | 15.8 ± 4.0 | 28.1 ± 5.9 |
| DHA₍₁₀₎ + CS | 10 | 60 | 14.7 ± 3.8 | 25.0 ± 5.2 |
| DBPs₍₅₎ + CS | 10 | 50^{a} | 11.7^{b} ± 3.1 | 13.2 ± 3.0 |
| DBPs₍₁₀₎ + CS | 10 | 40^{a} | 8.2^{b} ± 2.2 | 9.7 ± 2.0 |

^{a}p < 0.05 vs CS group (chi square test);
^{b}p < 0.01 vs CS group

TABLE 3
Effects of DBPs, EPA and DHA on CS-induced alteration of adrenal gland
ascorbic acid and corticosterone concentrations and plasma corticosterone
level

| | | Adrenal | Adrenal | Plasma |
|---|---|---|---|---|
| Groups | | ascorbic acid | corticosterone | corticosterone |
| (mg/Kg, p.o.) | n | (μg/100 mg) | (μg/100 mg) | (μg/dL) |
| Vehicle | 8 | 300.2 ± 38.4 | 4.4 ± 0.7 | 14.0 ± 1.3 |
| EPA₍₅₎ | 6 | 308.8 ± 28.7 | 5.7 ± 1.4 | 15.0 ± 1.6 |
| EPA₍₁₀₎ | 6 | 310.5 ± 26.0 | 5.2 ± 0.8 | 15.5 ± 1.1 |
| DHA₍₅₎ | 6 | 309.4 ± 30.4 | 4.8 ± 1.2 | 15.0 ± 0.9 |
| DHA₍₁₀₎ | 6 | 308.9 ± 27.4 | 5.5 ± 1.0 | 14.7 ± 1.0 |
| DBPs₍₅₎ | 6 | 309.1 ± 25.8 | 5.0 ± 1.3 | 15.7 ± 1.4 |
| DBPs₍₁₀₎ | 6 | 315.5 ± 25.5 | 5.4 ± 1.7 | 14.9 ± 1.5 |
| Chronic stress | 12 | 114.7 ± 16.0^{a} | 1.7 ± 0.5^{a} | 28.0 ± 3.0^{a} |
| (CS) | | | | |
| EPA₍₅₎ + CS | 6 | 138.5 ± 18.2 | 3.0 ± 1.4 | 17.9 ± 0.9^{b} |
| EPA₍₁₀₎ + CS | 6 | 144.2 ± 14.7^{b} | 2.9 ± 0.7^{b} | 18.3 ± 1.8^{b} |
| DHA₍₅₎ + CS | 6 | 140.7 ± 20.5 | 2.5 ± 1.0 | 22.5 ± 3.5^{b} |
| DHA₍₁₀₎ + CS | 6 | 148.0 ± 16.7^{b} | 2.8 ± 1.0^{b} | 17.9 ± 0.9^{b} |
| DBPs₍₅₎ + CS | 6 | 173.4 ± 18.2^{b} | 3.0 ± 1.4^{b} | 17.3 ± 0.7^{b} |
| DBPs₍₁₀₎ + CS | 6 | 198.5 ± 20.7^{b} | 3.2 ± 1.1^{b} | 16.8 ± 1.0^{b} |

^{a}p < 0.05 vs vehicle-control group;
^{b}p < 0.05 vs CS group

### EXAMPLE 16

### Antioxidant Effects of DBPs, EPA and DHA

A comparative study of the antioxidant defence provided by the three compounds, DBPs, EPA and DHA, was made. The results are given in Table 4. The reason for selection of this test (antioxidant-profile) is, that, agents that can regulate systemic production and interactions of reactive oxygen species, like singlet oxygen, superoxide radical and hydroxyl radical, can provide surveillance umbrella to living organisms against ‘oxidative stress’.

In this experiment, DBPs (1 and 2, Scheme-II, 1:1 mixture) in 0.1, 0.2 and 0.4 mM concentrations, were found to significantly L-DOPA (3,4-dihydroxyphenylalanine)-sparing (and, therefore, ¹O₂-quenching) effects. The singlet oxygen was generated on Rose Bengal-coated glass plates by illuminating with a 150-W spot-light at a distance of 30 cm, through water to filter infra-red light (Ghosal, S. and Bhattacharya, S. K. (1996). Antioxidant defence by shilajit, Indian J. Chem., 35B, 127-132). EPA (0.1-0.4 mM) and DHA (0.1-0.4 mM) showed only weak antioxidant effect in this test (Table 4).

TABLE 4
L-DOPA-sparing by ¹O₂-quenching effect^{a} of DBPs, EPA and DHA

| | | Percent inhibition of L- |
|---|---|---|
| | Concn of L-DOPA:test | DOPA oxidation^{b} |
| Group | compound (mM) | (mean ± SEM) |
| Control^{c} | —^{d} | 0 |
| DOPA + DBPs | 1:0.1 | 22.2 ± 2.1 |
| | 1:0.2 | 28.0 ± 1.8 |
| | 1:0.3 | 37.5 ± 3.9 |
| | 1:0.4 | 49.7 ± 4.0 |
| DOPA + EPA | 1:0.1 | 7.7 ± 1.3 |
| | 1:0.4 | 11.3 ± 3.2 |
| DOPA + DHA | 1:0.1 | 6.9 ± 1.8 |
| | 1:0.4 | 8.2 ± 0.9 |

^{a}mean of six to ten replicates
^{b}The concentration of unchanged L-DOPA, in solution, after exposure to ¹O₂ (30 min), in presence and absence of the test compounds, was estimated by HPTLC and HPLC using authentic marker.
^{c}L-DOPA in Pi buffer (pH 7.2)
^{d}indicates test compound absent; volume of reaction mixture, 200 μl.

Additionally, the facile transformation of EPA to DBPs in presence of Fe²⁺ (FIG. 2), and the subsequent stability of DBPs, in presence of the metal ion, suggest metal ion-captodative properties of DBPs (str. 16, Scheme-II) and the lack of it by the PUFAs.

### EXAMPLE 17

### Anti-Craving Effects of DBPs for Drugs of Abuse

Methylenedioxymethylamphetamine (MDMA) is used as a recreational drug of abuse. This illegal designer drug, related to amphetamine, is also known as ‘ecstasy’ and ‘love drug’ in abuser circles (Duxbury, A. J. (1993). Ecstasy—implications, Br. Dent. J 175, 38-45). As its abuse increased, making it the most popular recreational drug after cannabis, LSD and amphetamine, it became evident that MDMA was not the ideal safe non-toxic recreational agent as was claimed earlier and concerns have been raised about MDMA's addictive potential and neurotoxicity (Steele, T. D., Mc Cann, U. D. and Ricaurte, G. A. (1994). ‘Ecstasy’: pharmacology and toxicology in animals and humans, Addiction. 89, 539-55; Bhattacharya, S. K., Bhattacharya, A. and Ghosal, S. (1998). Anxiogenic activity of ‘Ecstasy,’ Biogenic Amines. 14, 217-37) (hereinafter referred to as “Bhattacharya et al. 1998”).

The clinical features of MDMA abuse toxicity and withdrawal syndrome suggest that this drug, like yohimbine, induces marked toxicity. The anxiety-inducing potential of MDMA was markedly reversed by DBPs, while EPA or DHA elicited only weak reversal effect (Table 5). This was determined according to a previously described method (Bhattacharya et al. 1998).

### EXAMPLE 18

### Open-Field Test

MDMA (5 and 10 mg/Kg, i.p.) produced a dose-related decrease in the number of squares crossed and rears, with concomitant immobility and increased defecation; these effects are qualitatively similar to those induced by yohimbine (2 mg/Kg, i.p. in 0.9% saline as the vehicle). DBPs (1 and 2, 1:1 mixture 10 mg/Kg, p.o. day-1, for 7 days) were administered prior to MDMA or yohimbine administration, on the 7th day, 1 hour after the last DBPs administration (p.o.). The results are incorporated in Table 5. Similar anti-anxiogenic effects were observed on pretreatment of MDMA, followed by DBPs.

TABLE 5
Effects of MDMA, yohimbine, DBPs, EPA and DHA on the open-field
test in rats (on anxiogenic test model).

| Groups | | Squares | | | Faecal |
|---|---|---|---|---|---|
| (mg/Kg) | n | crossed | Immobility | Rears | pellets |
| Vehicle- | 12 | 138.6 ± 9.8 | 42.4 ± 7.5 | 24.2 ± 5.4 | 4.4 ± 0.9 |
| control (0.9% | | | | | |
| saline) | | | | | |
| MDMA₍₅₎ | 8 | 111.2 ± 8.0 | 69.3 ± 6.1 | 14.3 ± 4.2 | 6.7 ± 0.5 |
| MDMA₍₁₀₎ | 8 | 74.7 ± 5.5 | 80.1 ± 7.0 | 11.2 ± 3.9 | 8.0 ± 2.2 |
| Yohimbine₍₂₎ | 8 | 90.8 ± 7.4 | 70.2 ± 4.9 | 12.8 ± 4.4 | 7.5 ± 1.2 |
| DBPs₍₁₀₎ | 10 | 128.1 ± 7.3 | 49.3 ± 8.4 | 18.2 ± 6.0 | 5.0 ± 0.8 |
| EPA₍₁₀₎ | 8 | 116.5 ± 9.9 | 70.1 ± 5.3 | 12.2 ± 4.8 | 6.8 ± 3.0 |
| DHA₍₁₀₎ | 8 | 122.1 ± 5.5 | 59.9 ± 7.2 | 14.0 ± 5.5 | 5.7 ± 4.1 |

The above findings suggest that ingestion of DBPs, would protect the recipients from the pre- and post-adverse anxiogenic effects of and cravings for MDMA and yohimbine-type drugs of abuse. EPA or DHA would not be truly effective for this purpose. There is evidence that presynaptic serotonergic, but not dopaminergic, mechanisms are involved in the enactogen-like discriminative stimulus properties of MDMA. MDMA increases the number of rat brain 5-hydroxytryptamine 5-HT_{1A} receptors and induces increased release of 5-HT from presynaptic terminals. The MDMA-withdrawal syndrome includes this increased 5-HT release activity in rats. Post-treatment of DBPs, but not EPA or DHA, completely prevented this adverse effect in MDMA-treated rats.

### EXAMPLE 19

### Hematinic Effect of DBP-Dimer

The significant hematinic effect of iron-complex (16) of 6 has been determined according to a previously described procedure (Ghosal, S., Mukhopadhyay, B. and Bhattacharya, S. K. (2001). Shilajit: a rasayan of Indian Traditional Medicine, Molecular Aspects of Asian Medicine, Vol. 1, PJD, Westbury, N.Y., 425-444).

The effect of administration (p.o.) of DBP-iron complex (16, iron:ligand, 1:4 mM ratio), for 7 days, to anemic albino rats, on their haemoglobin level is shown (Table 6).

TABLE 6
Effects of DBP-dimer-iron complex on level of hemoglobin in anemic rats

| | | Dose (mg/Kg | Haemoglobin | |
|---|---|---|---|---|
| Group and | | b.w., p.o. × 7 | (g/dL), before | |
| treatment | n | days) | treatment | After treatment |
| Group 1. | 10 | — | 6.08 ± 0.41 | 6.33 ± 0.52 |
| (control, 0.3% | | | | |
| CMC | | | | |
| suspension) | | | | |
| Group 2 (16) | 8 | 150 (iron, 5 mg) | 6.73 ± 0.50 | 9.88^{a} ± 0.26 |
| Group 3 | 10 | 150 (iron, 30 mg) | 7.06 ± 0.34 | 8.02^{b} ± 0.78 |
| (Fefol)^{c} | | | | |

^{a}p < 0.01 compared to Group 1;
^{b}statistically insignificant increase compared to Group 1;
^{c}ferrous sulphate, 150 mg capsule containing 30 mg iron

Pharmaceutical/Nutritional Formulations

### EXAMPLE 20

### Tablets and Capsules of the Invention

| Ingredient | Quantity per Tablet/Capsule | |
|---|---|---|
| 1. DBPs or their conjugates | 0.05-50% | by weight |
| 2. Avicel pH 101 | 200.00 | mg |
| 3. Starch 1500 | 189.00 | mg |
| 4. Stearic acid, N.F. (powder) | 8.60 | mg |
| 5. Cab-O-Sil | 2.00 | mg |

Note:
The target weight of tablet/capsule is 400 mg; Avicel pH 101 and Starch may be adjusted suitably to reach the target weight. The blended material can be filled into appropriate capsules.

### EXAMPLE 21

### Anti-Stress Support Tablets/Capsules of the Invention

| Ingredient | Quantity per Tablet/Capsule |
|---|---|
| 1. DBPs or their conjugates | 0.05-50% by weight |
| 2. Cellulose | q.s. |
| 3. Magnesium stearate | q.s. |
| 4. Gelatin | q.s. |

### EXAMPLE 22

### Cardio-Vascular Support Tablets of the Invention

| | | Quantity per Tablet/Capsule | |
|---|---|---|---|
| Ingredient | | | |
| 1. | DBPs or their conjugates | 0.5-30% | by weight |
| 2. | Vitamin A (Beta Carotene) | 45,000 | IU |
| 3. | Vitamin B-1 (Thiamin) | 25 | mg |
| 4. | Inositol Hexanicotinate | 50 | mg |
| 5. | Vitamin B-6 (Pyridoxine HCL) | 25 | mg |
| 6. | Vitamin B-12 (Cyanocobalamin) | 500 | mcg |
| 7. | Folic Acid | 800 | mcg |
| 8. | Vitamin C (Magnesium Ascorbate) | 150 | mg |
| 9. | Vitamin E D-alpha Tocophery (Natural) | 400 | IU |
| 10. | Copper (Sebacate) | 750 | mcg |
| 11. | Magnesium (Ascorbate, Taurinate, and | 30 | mg |
| | Oxide) | | |
| 12. | Potassium (Citrate) | 10 | mg |
| 13. | Selenium (L-Selenomethionine) | 200 | mcg |
| 14. | Silica (from 400 mg of Horsetail | 10 | mg |
| | Extract) | | |
| Other Ingredients and Herbs: | | | |
| 15. | Coenzyme Q10 (Ubiquinone) | 10 | mg |
| 16. | L-Carnitine L-Tartrate | 50 | mg |
| 17. | Hawathorn Berry Extract | 40 | mg |
| 18. | Grape Seed Extract | 10 | mg |
| 19. | L-Proline | 50 | mg |
| 20. | L-Lysine (HCL) | 50 | mg |
| 21. | N-Acetyl Glucosamine | 50 | mg |
| 22. | Bromelain (2,000 GDU per g) | 120 | mg |
| 23. | Taurine (Magnesium Taurinate) | 50 | mg |
| 24. | Inositol (Hexanicotinate) | 10 | mg |

### EXAMPLE 23

### Multi-Vitamin & Mineral Supplement Tablets of the Invention

| Ingredient | | Quantity per Tablet | |
|---|---|---|---|
| 1. | DBPs or their conjugates | 0.5-30% | by weight |
| 2. | Vitamin A (beta carotene) | 25,000 | IU |
| 3. | Vitamin A (palmitate) | 10,000 | IU |
| 4. | Vitamin B-1 (Thiamin Nitrate) | 10 | mg |
| 5. | Vitamin B-2 (Riboflavin) | 10 | mg |
| 6. | Inositol Hexanicotinate, Niacinamide & | 20 | mg |
| | Niacin | | |
| 7. | Vitamin B-5 (Calcium D-Pantothenate) | 10 | mg |
| 8. | Vitamin B-6 ((Phyridoxine HCL) | 10 | mg |
| 9. | Vitamin B-12 (Cyanocobalamin) | 200 | mcg |
| 10. | Biotin | 500 | mcg |
| 11. | Folic Acid | 800 | mcg |
| 12. | Vitamin C | 180 | mg |
| | (Magnesium, Manganese & Zinc | | |
| | Ascorbates) | | |
| 13. | Fat-Soluble Vitamin C | 20 | mg |
| | (from 476 mg of Ascorbyl Palmitate) | | |
| 14. | Vitamin D-3 (Cholecalciferol) | 400 | IU |
| 15. | Vitamin E D-alpha Tocopheryl | 600 | IU |
| | (Natural) | | |
| 16. | Boron (Amino Acid Chelate) | 2 | mg |
| 17. | Calcium (Succinate, Carbonate, Malate) | 20 | mg |
| 18. | Copper (Sebacate) | 1 | mg |
| 19. | Iodine (from Kelp) 150 mcg, | 150 | mcg |
| | Magnesium (Ascorbate, Oxide, | | |
| | Succinate) | | |
| 20. | Manganese (Ascorbate) | 30 | mg |
| 21. | Molybdenum (Amino Acid Chelate) | 300 | mcg |
| 22. | Potassium (Succinate, alpha- | 10 | mg |
| | Ketoglutarate) | | |
| 23. | Selenium | 250 | mcg |
| | (L-Selenomethionine & Sodium | | |
| | Selenite) | | |
| 24. | Zinc (Zinc Monomethionine & | 10 | mg |
| | Ascorbate) | | |

Other Ingredients and Plant antioxidants: N-Acetyl Cysteine, Succinic Acid (Free Form), Choline (Bitartrate), Inositol (Hexanicotinate and Inositol), N-Acetyl Glucosamine, DMAE (Bitartrate), N-Acetyl L-Tyrosine, Coenzyme Q10, Alpha-Lipoic Acid, Quercetin, Milk Thisle Seed Extract, Grape Seed Extract, Ginkgo Biloba, Bilberry Extract.

### EXAMPLE 24

### Anti-Diabetic Support Tablets/Capsules of the Invention

| Ingredient | Quantity per Tablet/Capsule | |
|---|---|---|
| 1. DBPs or their conjugates | 0.5-30% | by weight |
| 2. Vitamin B-6 (as Pyridoxine HCI) | 10 | mg |
| 3. L-Arginine | 50 | mg |
| 4. L-Lysine Monohydrochloride | 50 | mg |

| | |
|---|---|
| 5. Cellulose | q.s. |
| 6. Magnesium stearate | q.s. |
| 7. Gelatin | q.s. |

### EXAMPLE 25

### Weight Loss Support Tablets of the Invention

| Ingredient | Quantity per Tablet/Capsule | |
|---|---|---|
| 1. DBPs or their conjugates | 0.5-30% | by weight |
| 2. Garcinia Cambogia Extract | 60 | mg |
| 3. Bitter Orange Peel Standardized Extract | 20 | mg |
| 4. Green Tea | 10 | mg |
| 5. Cayenne | 15 | mg |
| 6. Mustard Seed | 10 | mg |
| 7. Ginger Root | 10 | mg |
| 8. Piper nigrum | 10 | mg |
| 9. Acetyl L-Carnitine | 10 | mg |
| 10. Niacinamide | 10 | mg |
| 11. Vitamin B-6 (Pyridoxine HCl) | 10 | mg |

### EXAMPLE 26

### Chewable Tablets of the Invention

| | | Composition |
|---|---|---|
| Ingredient No. | Ingredient | (% w/w) |
| 1. | DBPs or their conjugates | 0.5-30 |
| 2. | Sodium ascorbate, USP | 12-35 |
| 3. | Avicel pH 101 | 5-15 |
| 4. | Sodium saccharin, N.F. (powder) | 0.56 |
| 5. | DiPac | 10-30 |
| 6. | Stearic acid, N.F | 2.50 |
| 7. | Imitation orange flavor | 1.00 |
| 8. | FD&C Yellow#6 dye | 0.50 |
| 9. | Cab-O-Sil | 0.50 |

Procedure: Blend all the ingredients, except 6, for 20 min. in a blender. Screen in 6 and blend for an additional 5 min. Compress into tablets using 7/16-in standard concave tooling.

### EXAMPLE 27

### Syrup of the Invention

| Ingredient No. | Ingredient | Quantity per 100 mL |
|---|---|---|
| 1. | DBPs or their conjugates | 0.5-30% by volume |
| 2. | Excipients | q.s |

### EXAMPLE 28

### Oral Liquid of the Invention

| Ingredient | Quantity per 100 ml |
|---|---|
| 1. DBPs or their conjugates | 0.5-30% by volume |
| 2. Purified Water | q.s. |
| 3. Excipients: Preservatives, stabilizers, | q.s. |
| sweetners, flavors, colors, etc. | |

### EXAMPLE 29

### Snack Bar of the Invention

| In- | | |
|---|---|---|
| gre- | | |
| dient | | Quantity |
| No. | Ingredient | per 1 Kg |
| 1. | DBPs or their conjugates | 0.5-30% |
| | | by weight |
| 2. | Nutrition Blend: Calcium (Tricalcium Phosphate and | q.s |
| | Calcium Carbonate), Magnesium (Magnesium | |
| | Oxide), Vitamin A, Vitamin C, Vitamin D-3, | |
| | Vitamin B-1 (Thiamin), Vitamin B-2 (Riboflavin), | |
| | Vitamin B-6 (Pyridoxine), Vitamin B-12 | |
| | (Cyanocobalamin), Natural Vitamin (Acetate), | |
| | Niacin, Biotin, Pantothenic Acid, Zinc, Folic Acid, | |
| | Vitamin K, Selenium. Other Ingredients: Protein | |
| | Blend (Soy protein isolate, Hydrolyzed collagen, | |
| | Whey protein isolate, Calcium/Sodium Caseinate), | |
| | Glycerine, Polydextrose (fiber), Water, Cocoa Butter, | |
| | Natural Coconut Oil (non-hydronated), Coconut, | |
| | Cellulose, Cocoa Powder, Olive Oil, Lecithin, | |
| | Natural and Artificial Flavor, Maltodextrin, Guar | |
| | Gum, Citric Acid (Flavor Enhancer), Sucralose | |

### EXAMPLE 30

### Cereal with the Invention

| Ingredient | | Quantity |
|---|---|---|
| No. | Ingredient | per 1 Kg |
| 1. | DBPs or their conjugates | 0.5-30% |
| | | by weight |
| 2. | Excipients: Whole Grain Oats, Oat Bran, | q.s |
| | Sugar, Modified Corn Starch, Brown Sugar | |
| | Syrup, Salt, Calcium Carbonate, | |
| | Trisodium Phosphate, Wheat Flour, | |
| | Vitamin E (Mixed tocopherols), Zinc & Iron | |
| | (Mineral nutrients), Niacinamide (A B | |
| | Vitamins), Vitamin B6 (Pyridoxine Hcl), | |
| | Vitamin B2 (Riboflavin), Vitamin B1 | |
| | (Thiamin Mononitrate), Vitamin A | |
| | (Palmitate), Vitamin A B (Folic acid), | |
| | Vitamin B12, Vitamin D | |

### EXAMPLE 31

### Beverage with the Invention

| Ingre- | | |
|---|---|---|
| dient | | Quantity per |
| No. | Ingredient | 500 mL |
| 1. | DBPs or their conjugates | 0.5-30% |
| | | by volume |
| 2. | Excipients: Filtered Water, Food Starch- | q.s |
| | Modified, Citric Acid, Bitter Orange, Green Tea | |
| | Extract, Maltodextrin, Whey Protein Isolate, | |
| | High Fructose Corn Syrup and/or Sucrose and/or | |
| | Sugar, Sodium Benzoate, Caffeine, Niacin, | |
| | Glycerol Ester of Wood resin, Flavors, Colors | |

Veterinary Formulations

### EXAMPLE 32

### Chewable Tablets of the Invention

| Ingredient No. | Ingredient | Composition | |
|---|---|---|---|
| 1. | DBPs or their conjugates | 0.5-30% w/w | |
| 2. | Calcium (from calcium phosphate) | 600 | mg |
| 3. | Phosphorus (from calcium phosphate) | 470 | mg |
| 4. | Vitamin C | 10 | mg |
| 5. | Vitamin A | 750 | I.U. |
| 6. | Vitamin D3 | 400 | I.U. |

| | | |
|---|---|---|
| 7. | Excipients | q.s. |

Note:
Administer free choice just prior to feeding, or crumble and mix with food

### EXAMPLE 33

### Vitamin Tablets of the Invention (Peanut Butter Flavor)

| Ingredient | Quantity per Tablet |
|---|---|
| 1. DBPs or their conjugates | 0.05-50% by weight |
| 2. Other Ingredients: | q.s. |
| Brewer's Yeast Powder, Garlic, Whey, | |
| Beef Liver, Peanut Butter, Silica Gel, | |
| Niacin, Riboflavin, Thiamine | |
| Mononitrate, Ascorbic acid | |

### EXAMPLE 34

### Granules of the Invention

| Ingredient | Quantity per 4 oz. |
|---|---|
| 1. DBPs or their conjugates | 0.05-50% by weight |
| 2. Other Ingredients: | q.s. |
| Potassium Gluconate, Wheat, Sucrose, | |
| Hydrolyzed Vegetable Protein, Silicone | |
| Dioxide, TBHQ (preservative) | |

### EXAMPLE 35

### Blood-building Powder of the Invention

| Ingredient | | Quantity per lb. |
|---|---|---|
| 1. | DBPs or their conjugates | 0.05-50% by weight |
| 2. | Other Ingredients: | q.s. |
| | Heme iron polypeptide, Niacin (Vitamin | |
| | B3), Vitamin E acetate, Riboflavin | |
| | (Vitamin B2), Thiamine (Vitamin B1), | |
| | Pyridoxine (Vitamin B6), Vitamin B12, | |
| | Copper Sulfate, Cobalt sulfate, Soybean | |
| | oil, Whey, Natural sweet apple and | |
| | molasses flavors | |

### EXAMPLE 36

### Liquid Capsules of the Invention

| Ingredient | | Quantity per Capsule |
|---|---|---|
| 1. | DBPs or their conjugates | 0.05-50% by weight |
| 2. | Other Ingredients: | q.s. |
| | Safflower Oil, Gelatin, Fish Oil, | |
| | Glycerin, Borage Seed Oil, Vitamin E, | |
| | Water | |

Note:
The capsules may be punctured and the liquid contents squeezed onto food, if desired.

### EXAMPLE 37

### Oral Liquid of the Invention

| Ingredient | | Quantity per 100 ml |
|---|---|---|
| 1. | DBPs or their conjugates | 0.05-50% by volume |
| 2. | Purified Water, Sugar, Sorbitol, | q.s. |
| | Polysorbate 80, Propylene glycol, | |
| | Peptones, Ferric ammonium citrate, | |
| | nicotinamide, Vitamin A and D3 | |
| | concentrate, d-panthenol, Thiamine Hcl | |
| | (Vitamin B1), alpha tocopheryl acetate | |
| | (Vitamin E), saccharine sodium, | |
| | Vitamin A palmitate, Pyridoxine Hcl | |
| | (Vitamin B6), Riboflavin 5′-Phosphate | |
| | sodium (source of Vitamin B2) | |
| 1. | DBPs or their conjugates | 0.05-50% by volume |
| 2. | Excipients: Preservatives, stabilizers, | q.s. |
| | sweeteners, flavors, colors, etc. | |

### EXAMPLE 38

### Suspension of the Invention

| No. | Ingredient | Quantity per each oz. | |
|---|---|---|---|
| 1. | DBPs or their conjugates | 0.10-50.00% | |
| 2. | Fat (Polyunsaturated) | 45% | |
| 3. | Carbohydrate | 33% | |
| 4. | Vitamin A | 500 | I.U. |
| 5. | Vitamin D3 | 40 | I.U. |
| 6. | Vitamin E | 3 | I.U. |
| 7. | Thiamine Hcl (Vitamin B1) | 0.15 | mg |
| 8. | Riboflavin 5′Phos Na (Vitamin B2) | 0.17 | mg |
| 9. | Pyridoxine Hcl (Vitamin B6) | 0.2 | mg |
| 10. | Ascorbic acid (Vitamin C) | 6.0 | mg |
| 11. | Nicotinamide | 2.0 | mg |
| 12. | Pantothenic acid | 1.0 | mg |
| 13. | Folic acid | 0.04 | mg |

| | | |
|---|---|---|
| 14. | Sodium Benzoate | 0.1% |

### EXAMPLE 39

### Injectable of the Invention

| Ingredient | Quantity per ml |
|---|---|
| 1. DBPs or their conjugates | 0.1-10% by volume |
| 2. Water for Injection, USP | q.s. |
| 3. Ingredients to maintain proper pH | q.s. |

1. A composition of stable metabolites of docosahexaenoic acid (DHA) and eicosapentaenoic acid (EPA) comprising of oxygenated dibenzo-α-pyrones (DBPs).

2. A composition according to claim 1, wherein the oxygenated dibenzo-α-pyrones (DBPs) are in the form of conjugates.

3. A composition according to claim 1 further comprising said oxygenated dibenzo-α-pyrones of formula (I) 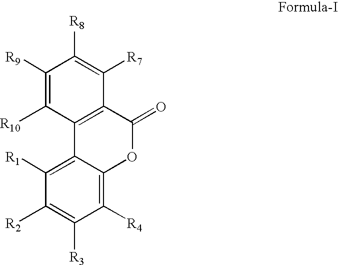
Wherein:
R₃ is selected from the group consisting of OH, O-acyl, O-aminoacyl, phosphocreatine;
R₈ is selected from the group consisting of H, OH, O-acyl, O-aminoacyl, phosphocreatine groups;
R₁, R₂, R₇, R₁₀ are independently selected from the group consisting of H, OH, O-acyl, O-aminoacyl, fatty acyl groups;
R₉ is independently selected from the group consisting of H, OH, O-acyl, O-aminoacyl, fatty acyl groups, and 3,8-dihydroxy dibenzo-alpha-pyrone (DBP) groups;
O-acyl groups are selected from saturated and unsaturated fatty acids having carbon chain lengths of about C₁₄ to C₂₄; and
O-aminoacyl groups are selected from methionine, arginine, glycine, alanine, threonine, serine, proline, and hydroxyproline.

4. The composition of claim 3 wherein R₉ is 3,8-dihydroxy dibenzo-α-pyrone (DBP) group,
said 3,8-dihydroxy dibenzo-α-pyrone (DBP) group is attached covalently at C-9.

5. The composition of claim 3 wherein said dibenzo-alpha-pyrones are 3-hydroxy and/or 3,8-dihydroxy dibenzo-alpha-pyrones.

6. The composition of claim 3 wherein said phosphocreatine is attached to the 3- or 8-hydroxyl functionality of said oxygenated dibenzo-alpha pyrone via an ester linkage.

7. A composition according to claim 3 further comprising transition and trace metal ions.

8. A composition according to claim 7 wherein said transition and trace metal ions are selected from the group consisting of iron, copper, calcium, zinc, magnesium, vanadium, molybdenum, and chromium metal ions.

9. A pharmaceutical, or veterinary, or nutritional formulation comprising the composition of claim 1 present in an amount of about 0.05% to about 50% by weight.

10. A pharmaceutical, or veterinary, or nutritional formulation comprising the composition of claim 2 present in an amount of about 0.05% to about 50% by weight.

11. A pharmaceutical, or veterinary, or nutritional formulation comprising the composition of claim 3 present in an amount of about 0.05% to about 50% by weight.

12. A pharmaceutical, or veterinary, or nutritional formulation comprising the composition of claim 5 present in an amount of about 0.05% to about 50% by weight.

13. A pharmaceutical, or veterinary, or nutritional formulation of claim 9 wherein said pharmaceutical or said veterinary or said nutritional formulation is administered to humans or animals in dose levels ranging from about 0.5 mg/day to about 500 mg/day.

14. A pharmaceutical, or veterinary, or nutritional formulation of claim 10 wherein said pharmaceutical or said veterinary or said nutritional formulation is administered to humans or animals in dose levels ranging from about 0.5 mg/day to about 500 mg/day.

15. A pharmaceutical, or veterinary, or nutritional formulation of claim 11 wherein said pharmaceutical or said veterinary or said nutritional formulation is administered to humans or animals in dose levels ranging from about 0.5 mg/day to about 500 mg/day.

16. A pharmaceutical, or veterinary, or nutritional formulation of claim 12 wherein said pharmaceutical or said veterinary or said nutritional formulation is administered to humans or animals in dose levels ranging from about 0.5 mg/day to about 500 mg/day.

17. The pharmaceutical, or veterinary, or nutritional formulation of claim 9 wherein said pharmaceutical, or said veterinary, or said nutritional formulation is administered at least once a day to humans or animals.

18. The pharmaceutical, or veterinary, or nutritional formulation of claim 10 wherein said pharmaceutical, or said veterinary, or said nutritional formulation is administered at least once a day to humans or animals.

19. The pharmaceutical, or veterinary, or nutritional formulation of claim 11 wherein said pharmaceutical, or said veterinary, or said nutritional formulation is administered at least once a day to humans or animals.

20. The pharmaceutical, or veterinary, or nutritional formulation of claim 12 wherein said pharmaceutical, or said veterinary, or said nutritional formulation is administered at least once a day to humans or animals.

21. A pharmaceutical formulation according to claim 9 wherein said pharmaceutical formulation is in the form of a tablet, syrup, elixir or capsule.

22. A pharmaceutical formulation according to claim 10 wherein said pharmaceutical formulation is in the form of a tablet, syrup, elixir or capsule.

23. A pharmaceutical formulation according to claim 11 wherein said pharmaceutical formulation is in the form of a tablet, syrup, elixir or capsule.

24. A pharmaceutical formulation according to claim 12 wherein said pharmaceutical formulation is in the form of a tablet, syrup, elixir or capsule.

25. A pharmaceutical formulation according to claim 9 wherein said pharmaceutical formulation contains about 0.5% to about 30% of said composition.

26. A pharmaceutical formulation according to claim 10 wherein said pharmaceutical formulation contains about 0.5% to about 30% of said composition.

27. A pharmaceutical formulation according to claim 11 wherein said pharmaceutical formulation contains about 0.5% to about 30% of said composition.

28. A pharmaceutical formulation according to claim 12 wherein said pharmaceutical formulation contains about 0.5% to about 30% of said composition.

29. A nutritional formulation according to claim 9 wherein said nutritional formulation contains about 0.5% to about 30% of said composition.

30. A nutritional formulation according to claim 10 wherein said nutritional formulation contains about 0.5% to about 30% of said composition.

31. A nutritional formulation according to claim 11 wherein said nutritional formulation contains about 0.5% to about 30% of said composition.

32. A nutritional formulation according to claim 12 wherein said nutritional formulation contains about 0.5% to about 30% of said composition.

33. A veterinary formulation according to claim 9 wherein said veterinary formulation contains about 0.5% to about 30% of said composition.

34. A veterinary formulation according to claim 10 wherein said veterinary formulation contains about 0.5% to about 30% of said composition.

35. A veterinary formulation according to claim 11 wherein said veterinary formulation contains about 0.5% to about 30% of said composition.

36. A veterinary formulation according to claim 12 wherein said veterinary formulation contains about 0.5% to about 30% of said composition.

37. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 9.

38. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 10.

39. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 11.

40. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 12.

41. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 9.

42. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 10.

43. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 11.

44. A method for treating ulcerogenic, inflammatory, stress, chronic stress, oxidative process, drug-induced cravings, anemia disorders, and for increasing a cognition effect of learning acquisition and memory retrieval comprising administering to a patient in need thereof a therapeutically effective amount of a composition according to claim 12.

45. A method of controlling synthesis and metabolism of arachidonate-derived prostaglandins comprising administering to a patient in need thereof a therapeutically effective amount of a composition according to claim 9.

46. A method of controlling synthesis and metabolism of arachidonate-derived prostaglandins comprising administering to a patient in need thereof a therapeutically effective amount of a composition according to claim 10.

47. A method of controlling synthesis and metabolism of arachidonate-derived prostaglandins comprising administering to a patient in need thereof a therapeutically effective amount of a composition according to claim 11.

48. A method of controlling synthesis and metabolism of arachidonate-derived prostaglandins comprising administering to a patient in need thereof a therapeutically effective amount of a composition according to claim 12.

49. A method of controlling synthesis and metabolism of arachidonate-derived prostaglandins comprising administering to a patient in need thereof a therapeutically effective amount of a composition according to claim 9.

50. A method of controlling synthesis and metabolism of arachidonate-derived prostaglandins comprising administering to a patient in need thereof a therapeutically effective amount of a composition according to claim 10.

51. A method of controlling synthesis and metabolism of arachidonate-derived prostaglandins comprising administering to a patient in need thereof a therapeutically effective amount of a composition according to claim 11.

52. A method of controlling synthesis and metabolism of arachidonate-derived prostaglandins comprising administering to a patient in need thereof a therapeutically effective amount of a composition according to claim 12.

53. A method of boosting energy comprising administering to a patient in need thereof a therapeutically effective amount of a composition according to claim 1.

54. A method of boosting energy comprising administering to a patient in need thereof a therapeutically effective amount of a composition according to claim 2.

55. A method of boosting energy comprising administering to a patient in need thereof a therapeutically effective amount of a composition according to claim 3.

56. A method of boosting energy comprising administering to a patient in need thereof a therapeutically effective amount of a composition according to claim 5.

57. A method of boosting energy comprising administering to a patient in need thereof a therapeutically effective amount of a composition according to claim 6.

58. A method of boosting energy comprising administering to a patient in need thereof a therapeutically effective amount of a composition according to claim 1.

59. A method of boosting energy comprising administering to a patient in need thereof a therapeutically effective amount of a composition according to claim 2.

60. A method of boosting energy comprising administering to a patient in need thereof a therapeutically effective amount of a composition according to claim 5.

61. A method of boosting energy comprising administering to a patient in need thereof a therapeutically effective amount of a composition according to claim 6.

62. A method of boosting energy comprising administering to a patient in need thereof about 0.5 mg/day to about 500 mg/day a composition according to claim 1.

63. A method of boosting energy comprising administering to a patient in need thereof about 0.5 mg/day to about 500 mg/day a composition according to claim 2.

64. A method of boosting energy comprising administering to a patient in need thereof about 0.5 mg/day to about 500 mg/day a composition according to claim 3.

65. A method of boosting energy comprising administering to a patient in need thereof about 0.5 mg/day to about 500 mg/day a composition according to claim 5.

66. A method of boosting energy comprising administering to a patient in need thereof about 0.5 mg/day to about 500 mg/day a composition according to claim 6.

67. A composition comprising the composition of claim 7 for the treatment of metal-deficient conditions.

68. A composition comprising the composition of claim 8 for the treatment of metal-deficient conditions.
